(19) United States
(12) Patent Application Publication
(10) Pub. No.: US 2003/0187587 A1
(43) Pub. Date: Oct. 2, 2003

(54) Database

(76) Inventors: Mark Swindells, Easton-on- the- Hill (GB); Janet Thornton, Herts (GB); David Jones, London (GB)

Correspondence Address: DARBY & DARBY P.C., P. O. BOX 5257, NEW YORK, NY, 10150-5257, US

(21) Appl. No.: 10/221,831

(22) PCT Filed: Mar. 14, 2001
(86) PCT No.: PCT/GB01/01105

(30) Foreign Application Priority Data
Mar. 14, 2000 (GB) 0006153.1

(51) Int. Cl. G06F 19/00; G06F 7/00; G06F 17/30; G01N 33/48; G01N 33/50

(52) U.S. Cl. 702/19; 707/1; 702/20

(57) ABSTRACT

The invention concerns methods and systems for predicting the function of proteins. In particular, the invention relates to databases in which details of sequence homologies, biological functions and structures that are shared between proteins of differing sequence have been compiled. The invention also relates to methods, systems and computer software that allows the prediction of protein function and structure and, optionally, the ligand binding properties of the proteins within such a database.

The present invention concerns methods and systems for predicting the function of proteins. In particular, the invention relates to databases in which details of sequence homologies, biological functions and structures that are shared between proteins of differing sequence have been compiled. The invention also relates to methods, systems and computer software that allows the prediction of protein function and structure and, optionally, the ligand binding properties of the proteins within such a database.

All cited documents are incorporated herein in their entirety.

There has recently been an unprecedented increase in the rate of generation of sequence data, due to advances in genetics and molecular biology and to the advent of large scale sequencing projects. Many experimental techniques needed to accelerate the generation of sequence data on a large scale have now been successfully scaled-up, allowing these strategies to be transported from the laboratory bench into an industrial context. In this environment, these techniques involve minimal human intervention and allow very rapid sequencing to take place at a relatively low cost.

As a result, over the last ten years, the volume of sequence data has continued to double every 18 months and this increase shows no sign of slowing pace. A significant increase in the early 1990s was associated with the deposit of tranches of Expressed Sequence Tags (ESTs). The main source of large deposit is now for completed microbial organisms or large regions of eukaryotic chromosomes.

The sequence information generated so far comes from a diverse selection of organisms. Whilst the genomes of complex organisms comprise tens of thousands or more genes, the less genetically-complex organisms have only around 500 genes. It is now appreciated that many genes possessed by apparently unrelated organisms are in fact derived by evolutionary divergence from common ancestors.

The amount of detail contained in sequence databases, such as GenBank (http://www.ncbi.nlm.nih.gov), the EMBL nucleotide data library at the European Bioinformatics Institute (http://www.ebi.ac.uk) and the DNA database of Japan (DDBJ) at the National Institute of Genetics (http://www.ddbj.nig.ac.jp), is immense and can cover such diverse information as the origin of the organism or chromosome from which the sequence data are derived and intron/exon information for each gene. The protein coding regions for each stretch of sequence of DNA may also be given (whether predicted or experimental).

Databases such as SWISS-PROT (http://expasy.hcuge.ch/) and PIR (http://pir.georgetown.edu/) devote themselves solely to protein sequence data. These databases also contain elements of additional information and include details such as the presence of N-terminal secretory signals and membrane-spanning regions.

The Protein Data Base (PDB) (http://www.rcsb.org/) contains information relating to all proteins whose 3D structures have been determined by x-ray crystallography, NMR spectroscopy or, to a lesser extent, electron crystallography. This database is much smaller than the DNA databases referred to above, although this database appears to be doubling in size every 18 months or so and now has well over 11,000 separate entries.

Although many genes with biologically important functions have now been cloned, sequenced and, to some extent, characterised biochemically, there are still a huge number of genes that have not yet been characterised in any way. Furthermore, for many of those genes that have been cloned, the function of the proposed gene product is unknown and cannot be predicted with any degree of certainty.

Although the available databases currently form the backbone of bioinformatics research, the vast amount of nucleotide sequence information that has been generated is presently of limited use, since the majority of these data are generally devoid of any experimentally-verified information about gene or protein structure or the functions of the encoded proteins. The practical exploitation of these sequence data is crucially dependent on the ability to identify the genes and the biological functions of the proteins that they encode.

Conventionally, research efforts directed towards elucidating the biological functions of the protein that a particular gene sequence encodes have involved sequence comparison with genes of known function, on the basis that similar or homologous genes or protein sequences are predicted to possess common ancestry and must therefore have similar functions.

A number of methods have been developed that attempt to extract functional information about a deduced amino acid sequence. These methods are mainly computer-based and utilise sequence alignment of either nucleotide or, more usually, protein sequences. However, these methods are generally limited by the extent of sequence similarity between the compared sequences. As the identity between the sequences decreases, these methods become increasingly erratic. Typical alignment methods include Smith-Waterman (Smith and Waterman, (1981) J Mol Biol, 147: 195-197), Blast (Altschul et al. (1990) J Mol Biol., 215(3): 403-10), FASTA (Pearson & Lipman, (1988) Proc Natl Acad Sci USA; 85(8): 2444-8) and, more recently, PSI-BLAST (Altschul et al. (1997) Nucleic Acids Res., 25(17): 3389-402). Assignment of function is based on the theory that significant sequence identity strongly predicts an evolutionary relationship from which function might be inferred.

This approach therefore fails when there is a lack of substantial sequence similarity between the sequence of interest and all other nucleotides or protein sequences. For sequences with at least 100 amino acids (300 nucleotides), this becomes a problem when sequence identity between compared proteins becomes lower than about 25-30%. It is estimated that as many as half of the proteins in a given genome exhibit similarities this low with proteins of known biological function. It is a further problem that for shorter sequences (and particularly fragments that can be generated by methods such as expressed sequence tag sequencing), matches with 30% or more sequence similarity can occur by chance. Furthermore, functional prediction based on small sequence signatures will be similarly unreliable. To be able to detect relationships beyond this barrier, we must look for other data. Conservation between primary amino acid sequences may frequently be low, yet the three dimensional structures of the related proteins may be surprisingly conserved. It is often the case that the overall tertiary structure of a protein is conserved, yet the primary sequence is significantly divergent.

Various solutions to this problem have been proposed. The goal of these methods is to improve the ability to detect distantly-related proteins and also to differentiate between true and false positives. However, there remains a great need for improved bioinformatics prediction platforms that are capable of predicting the biological functions of proteins with a high degree of confidence in an integrated manner. The present invention fulfils this need.

According to the invention there is provided a method of compiling a database containing information relating to the interrelationships between different protein and/or nucleic acid sequences, said method comprising the steps of:

a) integrating data from one or more separate sequence data resources into a combined database;

b) comparing each query sequence in the combined database with the other sequences represented in the combined database to identify homologous proteins or nucleic acid sequences;

c) compiling the results of the comparisons generated in step b) into a database; and

d) annotating the sequences in the database.

A database generated according to the method of the invention consists of an integrated data resource containing information generated from an all-by-all comparison of protein or nucleic acid sequences. The aim behind the integration of these sequence data from separate data resources is to combine as much data as possible, relating both to the sequences themselves and to information relevant to each sequence, into one integrated resource. All the available data relating to each sequence is thus integrated together to make best use of the information that is known about each sequence and thus to allow the most educated predictions to be made from comparison of these sequences. The annotation that is generated in the database and which accompanies each sequence entry imparts a biologically-relevant context to the sequence information.

In the case of a database of protein sequence information, the principal applications of the database of the invention are in pharmaceutical research. The database provides an immensely powerful and sophisticated resource that can be used to validate putative drug targets, and to identify new drug targets and drugs.

For example, for target validation, in many cases, experimental techniques have been used to identify proteins that are responsible for certain disease phenotypes. However, more information about the protein is required before it can be confirmed as a suitable target for drug design. The relational database of the invention can be used to allow prediction of, for instance, the structure and function of a novel protein in order to assess its potential as a drug target.

Furthermore, using the database, potential toxicities can be screened for. For example, if a bacterial protein sequence is found to be also present in humans, this suggests that an antimicrobial drug raised against that protein might be toxic to humans. The techniques documented herein can be used to prioritise candidate drug targets for development.

The database can also be used for target discovery, since its data can be mined to search for potential drug targets. For example, a user may search for new examples of sequences that belong to a clearly defined family of proteins, or identify conserved domains in a variety of different sequences and organisms. If the precise function of these domains is unknown, it can be generally be discovered by looking in the database at the properties of the proteins in which these domains occur.

Using a slightly different strategy, all sequences in the database that have a feature of pharmaceutical interest, such as a specific DNA-binding domain, may be identified. Proteins may also be identified that are related to a known drug target, in order to find fresh applications for an established drug series.

The database can also be used for drug discovery. Several human proteins with therapeutic potential have been identified from database searches. The relational database can be mined as discussed herein for known protein classes that may be good drugs, such as hormones, growth factors and cytokines.

In the case of nucleic acid sequences, a database according to the invention will be of immense value in assessing the evolutionary relationships between different sequences. Homologies may also be investigated between non-coding portions of nucleic acid, such as promoter regions, enhancer regions, and so on.

The database may contain both protein and nucleic acid sequences. This may be for completeness, so that, for example, when a protein of interest is identified in the database by a user, the encoding nucleic acid sequence may be accessed as required.

Inclusion of nucleic acid sequences may also facilitate the generation and comparison of the protein sequences that these sequences encode. For example, as a database is updated over time to reflect the discovery of new nucleic acid sequences, these new sequences could be checked against nucleic acid sequences already integrated into the database.

Sequences already incorporated into the database would thus be screened out to ensure that there is no duplication of protein sequence data in the database.

By sequence data resource is meant any database containing information relating to protein sequence data. Such a data resource may be a primary database or a secondary database. In the terminology used herein, the terms “primary” and “secondary” refer to the level of data that is contained in each database. It will be appreciated that any primary database, public or private, available now or in the future, will be equally applicable to the system of the invention. Ideally, all available information should be accessed for inclusion in the combined database. However, the more databases that are searched, the higher the chance of including redundant information that will need to be dealt with comprehensively by the system.

Conveniently, publicly available databases may be used, although private or commercially-available databases will be equally useful, particularly if they contain data not represented in the public databases. Primary databases are the sites of primary nucleotide or amino acid sequence data deposit and may be publicly or commercially available. Examples of publicly-available primary databases include the GenBank database (http://www.ncbi.nlm.nih.gov/), the EMBL database (http://www.ebi.ac.uk/), the DDBJ database (http://www.ddbj.nig.ac.jp/), the SWISS-PROT protein database (http://expasy.hcuge.ch/), PIR (http://pir.georgetown.edu/), TrEMBL (http://www.ebi.ac.uk/), the TIGR databases (see http://www.tigr.org/tdb/index.html), the NRL-3D database (http://www.nbrfa.georgetown.edu), and the Protein Data Base (http://www.rcsb.org/pdb).

Certain composite primary databases also exist that amalgamate a variety of different sequence resources. Examples include the NRDB (ftp://ncbi.nlm.nih.gov/pub/nrdb/README), and OWL (http://www.biochem.ucl.ac.uk/bsm/dbbrowser/OWL/). These databases provide protein sequence translations that have been taken from elsewhere and merged.

Commercially-available databases or private databases may also be used in the methods of the invention. Examples of commercially-available primary databases include PathoGenome (Genome Therapeutics Inc.) and PathoSeq (Incyte Pharmaceuticals Inc.).

Secondary databases derive extra value over primary databases by linking additional information to the contained sequences, for example, secondary motifs and functional annotation. It is this information which significantly contributes to the databases of the invention being such a useful resource, since the results generated by the all-by-all comparisons computed during the generation of the database are given a biologically-relevant meaning.

Examples of suitable secondary databases include the PROSITE (http://expasy.hcuge.ch/sprot/prosite.html), PRINTS (http://iupab.leeds.ac.uk/bmb5dp/print s.html), Profiles (http://ulrec3.unil.ch/software/PFSCAN_form.html), Pfam (http://www.sanger.ac.uk/software/pfam), Identify (http://dna.stanford.edu/identify/) and Blocks (http://www.blocks.fhcrc.org) databases.

Further to the primary and secondary databases, further databases containing any additional information of interest may be integrated, if required. Examples of such databases include the NCBI “Taxonomy” database (http://www.ncbi.nlm.nih.gov/Taxonomy/taxonomyhome.html) and the Expasy Enzyme Classification Database (http://www.expasy.ch/enzymes/index.html).

According to a second aspect of the present invention there is provided a method of compiling a database containing information relating to the interrelationships between different protein sequences, said method comprising the steps of:

a) integrating protein data from one or more separate sequence data resources and/or one or more structural data resources into a combined database;

b) comparing each query protein sequence in the combined database with the other protein sequences represented in the combined database to identify homologous proteins using, for each query sequence:

i) one or more pairwise sequence alignment searches,

ii) one or more profile-based sequence alignment searches;

iii) one or more threading-based approaches;

c) compiling the results of the comparisons generated in step b) into a database; and

d) annotating the sequences in the database.

Conveniently, information from the primary databases GenBank, SWISS-PROT and PDB may be integrated into the database. In this embodiment of the invention, structural data from the PDB are integrated with the sequence data from GenBank and SWISS-PROT to form a combined sequence and structural database. The PROSITE and PRINTS databases are used as secondary database sources, as their accompanying annotation is of considerable use. Additionally, entries from the Taxonomy and Enzymes databases are cross-referenced against all entries in the GenBank, SWISS-PROT and PDB databases.

In one aspect of this embodiment of the invention, the integration step a) in this method may include an extra preliminary step of incorporating nucleic acid data, for example, from a database such as GenBank. These data may be translated into protein to ensure that the available data for inclusion in the database are as complete as possible. Alternatively, these nucleic acid data may simply be linked to the relevant protein entries to provide a form of annotation that may accessed on demand.

In a preferred embodiment of the invention, when information from the GenBank database is incorporated into the database, each entry should be linked against the Enzyme and Taxonomy entries referred to above.

SWISS-PROT is a primary sequence database of proteins for which detailed annotations (such as descriptions of the function of a protein, its domain structure, post-translational modifications, variants, etc) have been compiled. The database also has minimal redundancy (i.e. large numbers of essentially similar sequences such as immunoglobulins have only a limited number of entries logged).

In SWISS-PROT, as in most other sequence databases, two classes of data can be distinguished: the core data and the annotation. For each sequence entry the core data consists of the sequence data, bibliographical references and the taxonomic data describing the biological source of the protein, The annotation includes descriptions of function as well as post-translational modifications domains and disease associated data. Preferably, the SWISS-PROT entries are linked against the taxonomy entries in a database according to the invention.

The PROSITE data resource is a key database of familial data supporting comprehensive manual annotation. The main features of this database are a set of regular expressions and profiles that when applied to sequences enable the user to determined whether a protein of interest belongs to a particular family or not. Comprehensive annotation enables sequences of unknown function to be scanned for hits to any regular expression or profile and the supporting annotation used to determine their likely function. PROSITE regular expressions and profiles may be used to search for matches against every sequence entered into the database.

PROSITE entries only contain a limited number of record types. The power of the PROSITE database comes from being able to relate annotation in the documentation file to a sequence by applying the supplied regular expressions and profiles. In a preferred embodiment of the invention, sequences entered into the database are compared directly against regular expressions and profiles parsed from the PROSITE database. The precalculated results can then be viewed by a user upon request through an appropriate interface.

The degree of information that can be obtained by linking PROSITE regular expressions to protein sequences in the combined database varies according to the complexity of the motif. For instance, the short GxGxxG motif is used in many NAD and FAD binding proteins to bind phosphate but is not exclusive to these proteins. Its identification therefore indicates the possibility of an NAD or FAD binding domain in any protein where a match is found, but is not confirmatory. By contrast, longer and more complex motifs are better at identifying a particular type of protein but a highly complex motif may yield few if any matches. There is therefore a trade-off between widely-matching but relatively non-specific simple motifs, versus more rare but highly specific matches with complex motifs.

Furthermore, there is no numerical description of a chance occurrence provided with each regular expression generated. However, a numerical description may be generated to indicate the degree of confidence that may be assigned to the likely correctness of a particular match. An example of such a categorisation which has been derived from an analysis of PROSITE and SWISS-PROT databases together is as follows:

| Quality | Perceived accuracy |
|---|---|
| High | 95% |
| Medium | 85% |
| Low | 50% |
| Very low | non-meaningful* |

*Expectation of chance occurrence is greater than one per sequence

This estimate may be done, by, for instance, a manner similar to that published by Sternberg, 1991 (Nature, 349: p111). By including such a method of categorisation in the generation of a database according to the invention, significant matches to regular expressions contained in PROSITE are thus available for each sequence within the database.

For profiles, preferably the implementation is precisely as specified by PROSITE and uses the cut-offs recommended. In cases where more than one match is identified, and there is no overlap, all matches should be included. In cases where overlap exists, it may be expressed as a proportion of the length of the lower scoring alignment. In a particularly preferred embodiment, both hits are reported when this is less than 80% of the lower scoring sequence. When 80% or higher, only the higher scoring alignment is displayed.

As an alternative strategy, the hits reported to PROSITE for SWISS-PROT could be parsed directly, but this would mean that sequences from other databases would not be annotated.

PRINTS is another major resource of family-based information featuring comprehensive annotation and a means to relate family-based annotation to individual sequences. Here the mechanism is a fingerprint rather than a regular expression or profile. These may be applied in an automated manner to sequences or simply read directly from each precalculated PRINTS entry, although the latter will only cover the SWISS-PROT and TrEMBL databases. PRINTS data files are slightly larger than PROSITE entries, primarily because they contain information on partial hits to fingerprints as well as information on how the final fingerprint was determined from an initial starting model. Preferably, only sequences with complete fingerprints are recorded.

Advantageously, the PRINTS entries should be linked against as many sequence codes as are common between the PRINTS entries that the primary databases loaded into a database according to the invention.

Furthermore, if included in a database according to the invention, it is advantageous to link the ENZYMES entries against the relevant information from the SWISS-PROT database.

Furthermore, if information from the database entries are also cross-referenced in the database against the taxonomy entries. The taxonomy assignments used are those from Steve Bryant at NCBI to map tax IDs to PDB chains (ftp://www.ncbi.nlm.nih.gov/mmdb/pdbeast/table).

Advantageously, protein structure files are also loaded into the database. As discussed above, protein structure files may be incorporated from any convenient database, either private, commercially-available or public. At present, the PDB resource is the most convenient. Indeed, protein structure information is conventionally presented in the form of actual “PDB” files. Protein structure files may therefore be incorporated into the database in this format, or, in fact, any other format, if desired.

The inventors have identified a growing number of inconsistencies in PDB files as more data continues to be released to the research community in an unreviewed form and consider that the value of these data may be very much improved if various checks are performed on the files, such that the files become “cleaned” of inconsistent or erroneous information. These steps are important because out of the 11,800 or so PDB files available at the time of writing, many contain errors, due to carelessness in the preparation of the original data files themselves.

Accordingly, in a particularly preferred embodiment of the invention, protein structure files are processed in an initial “cleaning” step before their incorporation into the database. This step may be performed for protein structure files in any file format. Preferably, this cleaning step may be performed using a program referred to herein as the pdb2xmas program and discussed in detail in section 1.1.1.2.1 below. This particular program is believed to be able to parse all PDB files successfully (including Level 1 releases) or at least to identify errors in the files and mark them for manual correction. This conversion program identifies errors in the PDB files and automatically corrects them using a number of distinct checks in order to provide high quality validated data that are stereochemically sound.

One example of a check that is particularly relevant involves the processing of protein structure files that contain data describing the ligand when bound in a ligand-protein complex. Of course, automated analyses of all ligands bound to protein structures can reveal a great amount about the binding/active sites of proteins; successful data processing is the first step in a complex process for displaying such information. However, while the amount of ligand data continues to grow, the records that describe where bonds occur in non-protein ligands are often incomplete or the records that generate bonds that contravene basic physical principles.

In addition to the problems of errors in the PDB files, the actual standard PDB format is considered to be imperfect and worthy of improvement. The current PDB format has two advantages, namely that it is simple and that it is relatively fast to read. Its chief failings are four-fold:

(i) the use of relatively unstructured comment regions;

(ii) inextensibility (no easy way to add additional information about an atom such as hydrophobicity, accessibility);

(iii) lack of structure in header records, such as bibliography and refinement information, repetition of residue-level information in each atom record; and

(iv) lack of enforcement of the standards and consistency checking by the PDB.

The inventors have therefore designed a novel flexible format that is easily extensible to allow the inclusion of additional data, that is simple and fast to parse and that is well structured. It will be appreciated that the method of structuring data into this format, the format itself, and programs capable of parsing data in this novel format may be used independently of the method of database generation described herein. These novel and inventive elements that are described herein in the context of generating a relational sequence database are considered to form a separate invention.

This novel format is referred to herein as the XMAS format. This format is discussed in more detail below (see section 1.1.1.2.1). As the skilled reader will appreciate, it is not essential to use this format for presenting protein structure data. Any format that provides high quality validated data is suitable for use in accordance with the present invention.

The description of the format contains two parts: (i) the syntax for creating files (i.e. like a description of extensible markup language (XML), hyper text mark-up language (html) or Abstract Syntax Notation One (ASN.1), and (ii) a data type definition for PDB files (i.e. a description of the required data content to describe a PDB file). This new format provides the advantage of the header defining a set of data columns that are specified in a data section and read very simply.

The data itself is read in a simple column format, much the same as in an ordinary PDB file (though in the case of a PDB file, there are no optional columns—all the columns are specified). Preceding the actual data, there is a block that defines the meaning of the columns and “append” tags are used to remove redundancy and add structure to the files. It is therefore trivial to add additional information to the data section such as per atom accessibilities, hydrophobicity values and so on.

For inclusion in the database, the residue accessibility for each residue in the structure is preferably determined and this information is appended to the protein structure file. In a preferred embodiment, this accessibility is determined for the protein structure in the XMAS format. It should be noted that it is the advantageous structure of the XMAS file format that makes it possible to append information such as this. In contrast, the conventional PDB format used for the presentation of protein structure data is inextensible and thus precludes such a possibility.

Preferably residue accessibility is assessed using the method published by Lee and Richards (1971), Journal of Molecular Biology, 55: 379-400. Other suitable methods are available, such as, for example, the MS program designed by Connolly (J Mol Graph June 1993; (2):139-41).

The secondary structure of the protein is also preferably determined and, again, this information is appended to the file. The secondary structure may be determined using one of any number of suitable algorithms. Preferably, the Kabsch-Sander algorithm is used (Kabsch W, & Sander C (1983) Biopolymers 22: 2577-2637), although other suitable methods are available (such as that of Frishman and Argos (1995) Proteins: Struct., Funct., Genet. 23: 566-579).

In a further step, the inter and intra-structure hydrogen interaction of the protein described in the protein structure file is preferably determined and this information appended to the file such that it is linked against the relevant protein sequences. This may conveniently be performed using the method described by Baker and Hubbard (1984) Progress in Biophysics and Molecular Biology, 44: 97-179. One alternative method is that described by McDonald and Thornton (1994) Journal of Molecular Biology 238: 777-793.

Protein-ligand interactions of the protein should preferably also be determined, if available. This may also conveniently be performed using Baker and Hubbard's method (op.cit.). This information should then be appended to the relevant file.

The protein structure files in their final format (preferably including PDB information, secondary structure information, residue accessibility data and inter-structure hydrogen interaction data) are then incorporated into the database.

After the initial step of loading sequences into the database, the data should preferably be processed such that the information from the collated primary databases is cross-referenced to one or more of the secondary databases. In a preferred embodiment of the invention, the data from the GenBank, SWISS-PROT and PDB databases is cross-referenced to the secondary database PROSITE.

Conveniently, collated sequence data from the primary databases in an initial step is converted to a unitary format to facilitate the subsequent analysis of data by programs external to the database itself. A suitable unitary format is the FASTA format, although any number of other formats could be adopted to allow subsequent analysis of the sequence data.

In order to reduce the load of comparisons that must be performed in order to compile the database according to the invention, redundant sequences should preferably be removed from further consideration. The redundancy of sequence data is a recurrent problem that has haunted the analysis of protein and DNA sequences during the development of this technology. Many entries in protein databases represent members of protein families or versions of homologous genes that are found in different organisms. Several groups may have submitted the same sequence and entries can therefore be more or less identical.

Accordingly, the data from all the separate databases is preferably parsed to identify matches that are identical or near-identical. This has the effect of removing redundancy from the database and ensures that the most information possible is derived from the available data whilst at the same time reducing unnecessary data processing to a minimum.

The reduction of redundancy in the method of the present invention may conveniently be achieved using a program developed by the inventors, termed “Dunce” and this is discussed in more detail below. However, redundancy may be reduced by any suitable method, such as, for example, the method described by Holm and Sander (1998) Bioinformatics 14: 423-429, or Bleasby et al., (1994) Nucleic Acids Research 22(17): 3574-3577.

The Dunce program reads one or more files containing protein sequence data in FASTA format and rewrites the data as a non-redundant data set in FASTA format to the standard output. Only input sequences that are not contained within other input sequences will be copied to the output. In addition, if multiple identical sequences occur in the input data, only the first one encountered will be a candidate for the output data set.

The Dunce program finds matches by splitting sequences into contiguous, non-overlapping fragments that are placed in a hash table. Then every possible (overlapping) fragment from each sequence is matched against the hash table to find possible matches. Candidate matches for a given sequence are found by comparing fragments against the bash table. If two fragments from different sequences match in the hash table, the complete sequences are checked against each other character by character.

Dunce has been written to run quickly with large numbers of input sequences, at the expense of requiring large amounts of memory. Using this program, over 400,000 sequences have been processed in 15 minutes on a Sun Ultra Sparc computer with 1 Gbyte of memory.

There is also the facility to specify that the Dunce program ignores a given number of internal differences and thus performs only approximate comparisons. A command line flag specifies a so-called “fuzz factor”, given a positive integer parameter that equals the number of individual residue differences within a sequence comparison which will be accepted before the comparison sequences are deemed to differ.

If the sequence being processed is found to be either identical to, or a subsequence of, one already in the hash table (which would thus be a “supersequence” of the sequence being processed), then this fact is recorded, and no further processing is done on this sequence. Processing continues with the next sequence in the input data set.

Alternatively, if any of the candidate matches is found to be a subsequence of this sequence, then that is recorded and for each subsequence found, all the corresponding fragments in the hash table are deleted.

Finally, if no identical or supersequences are found, this sequence is added to the hash table. In distinction to the checking stage above which used overlapping fragments, only contiguous, non overlapping fragments are actually added to the hash table.

The process is repeated sequentially for all the sequences in the input file(s). The Dunce program can also accept multiple input files. If a new sequence file or files become available, then it is possible to speed up the process of adding these to a file that is already non-redundant by means of an “update” flag given to Dunce at the run time. If this flag is given then the Dunce program will simply add the contiguous fragments of the non-redundant sequences to the hash table, without checking for any matches. If used correctly on a non-redundant file, then of course there would not have been any matches anyway. Only when processing reaches the second and subsequent files will Dunce start checking for hash table matches.

The update flag is only of use to speed up processing and then only when one file is already known to be internally non-redundant. When correctly used, it has no effect on the actual data that are output.

An example of an alternative algorithm that achieves the same task as that undertaken by Dunce consists of only putting a single fragment from each sequence into the hash table. For this to work, a two pass process is required, first putting a single fragment from each sequence into the hash table and then, on the second pass, comparing all the overlapping fragments from each sequence with the hash table. This reduces the number of entries in the hash table by a factor of roughly L_{A}/K where L_{A} is the average sequence length. This reduction is at the expense of a second pass.

There are variations on this theme, for example a three-pass variant, where the first pass builds a histogram of fragments and only the most unusual fragment from each sequence is added to the hash table. This will reduce the number of hits and subsequent full comparisons that are necessary on the final pass.

Details of the precise algorithm used in the preferred embodiment of the invention may be found in section 1.1.2.3 below.

Non-redundant sequences output by the redundancy-removal program are then loaded into the database. Once all desired sequences have been loaded into the database according to the invention, the database constitutes a huge resource of primary cross-inked data with preliminary annotation included.

For subsequent analysis of the sequences in the database, the inventors have found it preferable to mask sequences either known to recur frequently in sequences that are in fact unrelated, or having properties that are not amenable to the sensitive algorithms employed in the comparison steps. This is useful because conventional analysis programs tend to group proteins together incorrectly when they are confused by the compositional bias in particular regions of the sequence.

In a preferred embodiment of the present invention, the primary data are thus masked to remove regions such as transmembrane domains, signal peptides, coiled-coil regions and other regions of low complexity as well as transmembrane domains that are not amenable to sensitive searches, having a lower complexity that typical globular proteins that exist in an aqueous environment. Any number of masking protocols may be utilised, although the method of the present invention most preferably utilises those exemplified below.

One such region of low complexity is a signal sequence. In order for a protein to be secreted from the cell, a signal sequence is required. These tend to be short and located towards the N-terminal of the sequence. In general, their properties are quite similar to those of transmembrane proteins and therefore it can be particularly easy to confuse a signal peptide with the insertion helix of a transmembrane region. As a result, signal peptides should conveniently be masked off before transmembrane regions.

Specifically, to mask for signal peptides, knowledge is required of sequences for which the cleavage sites are known. The SWISS-PROT database contains this information for a number of the sequences contained within it, and these can be used to generate testing and training sets. One way of generating a negative set involves the selection of sequences found only in the nucleus or cytoplasm and that thus do not have a signal peptide region. To select a positive set, a method can be used such as that described by Nielsen et al., 1997 (Protein Engineering, 10: 1-6).

One such method involves the construction of log probability matrices containing a set of log-probability scores (one each for gram positive, gram negative and eukaryotes, since signal peptides have different chemical properties within these subdivision) of residue preferences over a residue window of convenient length about the cleavage sites (for example, the score may be applied to a residue at an offset of 0 in a (−25,+5) window. Graphs of the datasets containing cleavage sites may then be compared with the results for sequences where no signal peptides are present.

Also, the scoring matrix used in MEMSAT (Jones et al., (1994) Biochem. 33: 3038-3049) may be used to provide an additional score that detect membrane-like regions by scanning a 20 residue window along the first 70 residues of each sequence. The MEMSAT score is applied to all of the residues in the 20 residue wide window, with each residue taking the highest scoring window it lies in. A threshold value is used that gives a 1% false-positive rate of detection, looking for any regions that achieve a score of at least 6000. If more than one window exceeds this score, the highest position is recorded. These are hydrophobic regions that could be either a single transmembrane helix or a signal peptide. To determine whether or not this region is a signal peptide, the properties derived from Nielsen's set of SWISS-PROT-derived sequences may be applied (Nielsen et al., op.cit.) and the scan is started from the peak hydrophobicity score. If a score above a specific threshold is identified, this is accepted as a cleavage site.

The masking of sequences for low-complexity regions is preferably performed in three stages: local, windowed and complete sequence.

Local masking is designed to take out small regions of sequence which are of very low complexity (for example, ATSSSSSAAS). One simple, effective method is to use a sliding window and mask regions where the average occurrence of residues in that window is 3. For example, if the sequence is GGGGHHHHLLLL, then the average repeat would be 4 ((4+4+4)/3), if it was GGGGGGHHHHHH (or GHGHGHGHGHGH) the value would be (6+6)/2=6, and if it was AACCDDEEFFGG the value would be 2 (and not be masked). The skilled reader will appreciate that other window sizes and thresholds for masking may be used with similar results.

Both of the windowed and complete sequence masking stages are based on the probabilities of a residue occurring within a sequence or a window. These probabilities may be calculated from a non-redundant database of 270,000 sequences taken from GenBank.

For each residue type, the distribution of the residue may be assessed within whole sequence and within a window of, for example, 100 residues. Thresholds that represent distances of Standard Deviation values from the mean are then calculated. If the composition of either the sequence or the window for each residue type lies beyond a certain value for the given residue, such as the 4 or 5 S.D. cut-off, then that residue type is masked out from the entire sequence/window. The 4 or 5 S.D. cut-off was chosen as an advantageous convenient cut-off by comparing the error rate when performing sequence searches on a database containing both normal and reversed versions of a set of sequences which were masked by this method at different thresholds.

The term “Coiled-Coil” covers a characteristic that has only been detected relatively recently. An excellent example of these are leucine zippers. Leucine zippers do not tend to be in regions responsible for protein activity. In contrast, they appear to act most frequently as molecular zips, tying two molecules together. They have leucine residues at regular separations along the sequence. The replication terminator protein is an example of a protein whose function is dependent on the presence of a leucine zipper. This protein is only active in its dimeric form and dimerisation occurs by means of a leucine zipper.

The masking of coiled-coil regions may conveniently be performed using the method described by Lupas et al. (1991) Science 252:1162-1164. Most preferably, the version utilised in the method of the present invention uses the MTIDK matrix over a 21 residue window, without the extra weighting for coil positions ‘a’ and ‘d’. If a region has a probability score of greater than 50%, then it is masked.

Transmembrane sequences also include sequences of lower complexity, meaning that the natural amino acids occur with quite different frequencies in transmembrane sequences. More specifically, hydrophobic amino acids tend to occur far more frequently. Because of this, the chance of finding a sequence similarity by chance is higher for transmembrane sequences and, as a result, searches cannot be as sophisticated. In order to achieve good search results the hydrophobic regions are masked out during sensitive searches leaving the comparisons to depend on the loops between each transmembrane helix which are exposed to the solvent.

The masking of these regions may be performed by one of any number of algorithms specifically designed for this purpose. One possibility is to use the MEMSAT program that was described by Jones et al., 1994 (op. cit). This program has the advantage that it predicts the topology of a membrane protein.

In general, for a particular region to form a transmembrane helix, there must be at least one helix that is considerably hydrophobic. Using this algorithm, all cases are thus accepted where an exceptionally hydrophobic sequence has been identified. In addition, cases are also accepted where there is a reasonably hydrophobic primary insertion helix coupled with a strong overall topology prediction.

The MEMSAT program thus gives a list of potential candidates from transmembrane regions together with a score for each helix predicted to be in the membrane and the overall score, considering the likelihood of all helices predicted being present in a particular topology. In a preferred implementation of the invention, if the overall score is greater than 8.0, or if the overall score is greater than 3.0 and an individual region gets a score greater than 0.5, then the sequence is masked appropriately; otherwise it is left unmasked.

All residues that are masked in the individual masking steps are then masked off from consideration in subsequent analysis steps.

After the steps to reduce redundancy and the masking steps, a selected set of sequences exists out of the total sequences initially loaded into the database that define the set of sequences to be used in the subsequent analysis steps discussed below.

In step (b) of the method according to the present invention, each query protein sequence in the database is compared with the other selected protein sequences in the database to calculate relationships between the proteins and thus identify homologous proteins. For each query sequence, one or more pairwise sequence alignment searches and one or more profile-based sequence alignment searches and one or more threading-based approaches are used.

The aim of this aspect of the method is to make the greatest possible use of the enormous amount of primary data that has been incorporated into the database in step (a) discussed above, to generate a database that contains information relating to the interrelationships between different protein sequences that may be used to allow the prediction of proteins of unknown function.

There are now a significant number of pairwise alignment programs that align protein sequences. These programs vary as regards the types of alignment performed (local or global), the speed with which they are capable of operating, the amount of memory that they require for a given volume of sequence data and so on. Examples of well known alignment algorithms include Smith-Waterman (Smith and Waterman, (1981) J Mol Biol, 147: 195-197); Needleman and Wunsch, (1970) J Mol Biol, 48: 443-453), BLAST (Altschul et al., (1990) J Mol Biol, 215: 403-410); FASTA (Lipman and Pearson, (1985) Science, 227: 1435-1441), and gapped BLAST (Altschul et al., (1997) NAR, 25(17): 2289-2302). It is likely that further developments in this area of Bioinformatics that generate suitable alignment algorithms will continue as the technology develops in this area.

In a preferred embodiment of the invention, a pairwise local alignment procedure is used that is based on the gapped BLAST (Basic Local Alignment Search Tool) program. This is supplemented by profile-based searching and genome threading.

Using these techniques, pairwise all-to-all sequence similarity searches are performed on the selected sequences in the database. Any new sequences not represented in the public databases may be tested against the entire database as they are introduced using the same search algorithms.

In a preferred embodiment of the method of the invention, gapped BLAST is used to match each input sequence in turn, matching the sequence against all other selected sequences for areas of commonality.

In this aspect of the method, for each sequence, a pairwise search is performed against the database of potential matches, for sequences with similar portions to the subject sequence. Similarity is determined by statistical relevance, the threshold for which may be determined according to the requirements of the particular system. For example, in a preferred embodiment of this invention, statistical relevance is viewed as representing an E value cut-off of less than 0.001 using an effective search space for gapped BLAST of 9 billion. However, as the skilled reader will appreciate, other cutoffs using. different expected error rates could also be used.

Although gapped BLAST is a powerful first-pass approach to sequence analysis that will identify most of the selected sequences within a database that are related to the query sequence, some biologically significant relationships may still escape detection. Therefore, a modified form of BLAST is preferably used, termed PSI-BLAST, to increase further the search sensitivity. However, any suitable profile-based method may be used.

Rather than using standard pairwise alignment with a universal substitution matrix, PSI-BLAST adopts a profile-based approach. The initial pairwise gapped BLAST run identifies a number of sequences in the database that match the query sequence, and these are then used to construct a profile that captures the key features of the sequences as a group. The profile, rather than the initial query sequence, is then used by the BLAST algorithm to scan the database a second time. New sequences are identified, the profile is built up and the entire process can be iterated until no new sequences are identified. The results may be displayed as a series of sequences arranged in a manner analogous to a multiple alignment.

The profile that PSI-BLAST constructs takes the form of a position specific scoring matrix, which directly specifies a score for each amino acid substitution along the length of the sequence. The matrix has the same length as there are amino acids in the original query sequence and a depth of most usually 20 (with additional cells optionally being made available for undefined residues. For example, the annotation “X” can occur due to a previously poor definition at the DNA or protein sequencing stage) This gives the score for finding each of the 20 possible amino acids at each point along subject sequences within the database. The scores for each cell within the matrix are weighted according to the frequencies of the amino acids that occur at the equivalent point in the sequence.

In standard pairwise searches, the search protocol is symmetrical in that the same similarity score is derived irrespective of which sequence is the query and which is the target. In PSI-BLAST, however, comparisons between sequences may be carried out in both directions since a different profile, and thus a different pool of homologues, is likely to be accumulated depending on the precise nature of the initial query sequence. Therefore, in a preferred embodiment of this aspect of the invention, two-way profile-based alignment data are therefore generated for every sequence represented in the database. This means that every protein is used in turn as a query sequence, having its own profile generated as part of the iterative search procedure. As a result, every pair of proteins in the database will be compared twice but the profile used will probably be different (originating from a different sequence). It is therefore possible that a relationship is only identified by one of the comparisons due to inequalities between the profiles. In contrast, in conventional bioinformatics procedures, the researcher only normally sees the results from the search direction in which the protein of interest is the query sequence (i.e. the one that has a profile generated for it). An advantage of performing an all by all comparison is that it ensures that relevant relationships are provided for the user, even when a relationship was only identified by one of the two comparison directions. Accordingly, in a preferred embodiment of the invention, relationships that are only detected in one direction have their results duplicated, for example, in the appropriate Oracle table, by a post-processing step. These hits may be presented to the user as reverse hits with negative iterations if the direction of the search requested is not the direction that actually identified the relationship.

The results of the alignments are preferably extracted and reformatted into a unitary format that presents all of the relevant information generated. For example, in the embodiment of the invention referred to above, the PSI-BLAST results should be extracted and reformatted. A suitable format records the total number of iterations that the search performed; and for each sequence hit, presents:

(a) the name of each sequence hit;

(b) the length of the match;

(c) the hit “bit score” representing a score of the profile generated by this hit;

(d) the hit “e-value”, representing a normalization of the “bit score” and thus the confidence of the hit;

(e) the number of identical residues found in the match;

(f) the number of positive scoring residues found in the match;

(g) the index of the starting residue of the match in the subject sequence;

(h) the index of the ending residue of the match in the subject sequence;

(i) the index of the starting residue of the match in the sequence found;

(j) the index of the ending residue of the match in the sequence found; and

(k) the PSI-BLAST iteration in which the match was found.

A significant number of hits will be generated for each profile-based search that is performed. The inventors have thus found it preferable to cluster the results in order to reduce redundancy in their content, since a number of results are generally output that represent approximations of the same regions of sequence overlap. The inventors have designed a novel method to solve this problem, based on the application of a graph subset construction algorithm, with two nodes considered to be connected if a significant region of two alignments overlap. It will be appreciated that this method of reducing redundancy in the results of multiple alignments and selecting a single representative alignment out of multiple results may be used independently of the method of database generation described herein. This novel and inventive method is considered to form a separate invention and is the subject of a co-owned United Kingdom patent application.

The method is summarised as follows. However, the skilled reader will appreciate that any other suitable method, existing now or developed in the future, may be used for the reduction of redundant alignment information.

This method uses a clustering program to cluster sequence matches, that identifies related sequences produced in the alignment steps and assigns them to a particular family. The algorithm used describes a method for combining multiple results from one or more sequence database searches into a single result for each distinct ‘hit’. For example, when performing a database search using an iterative algorithm such as PSI-BLAST, the alignment and E-Value may change between iterations, but it still ‘describe’ the same basic region of similarity between the two sequences.

This algorithm is described below and provides an automated method for finding and producing these similar regions from sets of individual sequence alignments.

When two sequences are aligned, regardless of the algorithm used, the resultant values can be split into two groups. The first group contains those values that describe the location of the aligned region of the two sequences denoted A & B. These results can always be represented by four numbers, as gaps in the alignment are not taken into consideration.

The first two numbers of the first group describe the extent of the aligned region on sequence A, denoted as [F_{A}, T_{A}], and the second two describe the extent of the aligned region on sequence B, denoted by [F_{B}, T_{B}]

The second group contains those output values which are related to the score or scores produced by the alignment algorithm. For example, useful outputs from the PSI-BLAST algorithm include the E-Value and the iteration number.

To explain the rationale governing the decision as to whether or not any two alignments are combined into one, the representation shown in FIG. 1 may be used.

The horizontal axis represents the residue numbers from sequence A, and the vertical axis residue numbers from sequence B. It can be seen that if perpendicular lines are drawn from the position of four numbers representing the alignment, then that alignment region is represented by a rectangle.

In considering two alignments, and whether or not they can be combined into one, there are three possible cases.

In the first case (FIG. 2), the two regions are disjoint, and so the two alignments can be trivially rejected as candidates for being combined.

In the second case (FIG. 3), one region is completely enclosed within another. These two alignments are therefore suitable for merging, with the new representative being the larger of the two regions.

Finally there is the case where the two regions intersect (FIG. 4). The method of the invention decides whether or not these two regions should be merged, based on the area of the intersection. If this area is significant, then the two alignments are merged into one.

The threshold value that defines a significant overlap varies depending on the algorithm or method that is being used to generate the alignment. Using PSI-BLAST alignment results, a figure of 90% has been found to work well (if the area of intersection of the two regions is greater than or equal to 90% of the area of the smaller of the two regions, then the regions are merged).

The value of 90% can of course be varied to suit the particular requirements of the analysis being carried out, but this figure was chosen as it worked well for the combination of results generated by PSI-BLAST. However, this figure is an arbitrary value that can be modified by a user depending upon the algorithm that is used. Preferably, this value is set between 80 and 99%, more preferably, between 85 and 95%.

If the two regions are suitable for merging, then the combined region then becomes the bounding box of the two rectangles (represented by the dashed line in FIG. 4).

For separate alignments of two sequences, the method of the invention can be illustrated as follows. As discussed above, a first alignment between a query sequence A at positions [F_{A}, T_{A}] and a target sequence B at positions [F_{B}, T_{B}] may be represented graphically with the horizontal axis representing the residue numbers from sequence A, and the vertical axis representing the residue numbers from sequence B, such that a rectangular region marked by co-ordinates [F_{A}, F_{B}], [T_{A}, F_{B}], [T_{B}, F_{A}], and [T_{A}, T_{B}] represents a first region of alignment. A second alignment between the query sequence at positions [F′_{A}, T′_{A}] and the target sequence at positions [F′_{B}, T′_{B}] may also be represented graphically such that a rectangular region marked by co-ordinates [F′_{A}, F′_{B}], [T′_{A}, F′_{B}], [T′_{B}, F′_{A}] and [T′_{A}, T′_{B}] represents a second region of alignment. According to the invention, the first and second alignments are combined if there is a significant region of intersection between the two regions of alignment.

Preferably, the two regions are combined if the area of intersection of the two regions is greater than or equal to 80% of the area of the smaller of the two regions. More preferably, this value is set at between 85 and 99%, more preferably, between 85 and 95%.

In the case where there are multiple alignment regions, such as when there is one alignment generated from each iteration of a repeating algorithm such as PSI-BLAST, the above calculations must be repeatedly performed, continually merging alignments together until no more candidates for merging are found. Finally there will then be one alignment representative for each distinct alignment region of the sequences that can be found.

The method may thus be broken down into steps involving extracting the results of the alignment of two separate sequences using a repeating alignment algorithm, followed by merging the results together if there is a significant region of overlap between them.

In order to perform this procedure efficiently, a ‘subset construction’ algorithm may be used (see, for example, Object-Oriented Software Construction, Bertrand Meyer [ISBN: 0136291554]). This will minimise the number of comparisons that need to be done between alignment pairs.

It should be noted that the example shown in FIG. 2, in which one region is completely enclosed by another, has been shown as a completely separate case. However in reality, this is just a special case of two regions intersecting, in which the area of overlap must be greater than a certain proportion (for example, 90%) of the smaller rectangle. The reason for showing this example as a separate case is that it is much easier to calculate than the general case of partial overlap. Therefore, if all of the enclosed alignments are removed first, there are less alignments to compare afterwards. This has the effect of speeding up the calculation. Accordingly, in the method of the invention, the step of merging alignment results together is preferably performed in iterative steps, whereby each alignment that is completely subsumed by another alignment is merged with the larger alignment before overlapping alignments are considered.

This aspect of the invention therefore provides a method according to any one of the aspects described above, wherein said combining step comprises the sequential steps of:

i. combining alignment regions in which one alignment region subsumes another; and

ii. combining alignment regions that only partially overlap.

It should be noted that alignment values are independent of the merging procedure and can be changed to suit the particular application. In the case of merging results from PSI-BLAST, the values that have been found to be of particular interest were the iteration number and the E-Value combination. These were required for the first, best and last iterations in which an alignment occurred.

In a particularly preferred embodiment of the invention, when two regions are merged using the above criteria, the lowest and highest iteration/E-Value pair present in the two alignments are stored in the combined alignment, along with the lowest E-Value achieved by either of the two alignments together with the iteration number at which this was achieved.

In use, it has been found that the application of this algorithm to the results of a PSI-BLAST search which ran for 20 iterations can reduce the total number of hits to as little as one fiftieth of their original number.

The results of the hits are preferably extracted and reformatted into a unitary format that presents all of the relevant information generated. For example, in the preferred embodiment of the invention referred to above, the PSI-BLAST results should be extracted and reformatted. A suitable format records the total number of iterations that the search performed and for each sequence hit presents:

(a) the name of each sequence hit;

(b) a local hit number, such that this, grouped with the name of the sequence hit, are unique for a subject sequence;

(c) the length of the longest match in the cluster;

(d) the bit score of the hit with the “best” e-value;

(e) the hit “e-value”, representing the lowest e-value recorded over all the hits grouped;

(f) the count of identical residues for the hit with the “best” e-value;

(g) the positive score count of the hit with the “best” e-value;

(h) the lowest index of the starting residue of the matches in the cluster in the target sequence;

(i) the highest index of the ending residue of the matches in the cluster in the subject sequence;

(j) the lowest index of the starting residue of the matches in the cluster in the subject sequence;

(k) the highest index of the ending residue of the matches in the cluster in the subject sequence;

(l) the lowest PSI-BLAST iteration of the hits in the cluster;

(m) the e-value of the hit of the lowest PSI-BLAST iteration in the cluster; and

(n) the highest PSI-BLAST iteration of the hits in the cluster.

These results are then loaded into the database.

In order to facilitate analysis of the interrelationships exhibited between proteins, that are evident from the information contained within the database according to the invention, it has been found preferable to generate alignments using a multiple alignment program. The goal of such a method is to generate a concise, information-rich summary of related sequence data. Programs that perform such multiple alignments are known. One example includes Clustal W (Thompson et al., 1994, NAR, 22(22), 4673-4680). However, such programs work best on small sets of short sequences; with longer, multiple sequences, the time required for multiple alignment generation is too slow. Approximations used by PSI-BLAST to generate multiple alignments generally contain large and multiple gapped regions.

The inventors have thus designed a novel method of performing multiple sequence alignments, in which the alignments of supplied sequences are constructed in relation to a given sequence. Additionally, this method includes a feature for constraining this algorithm to produce alignments that are consistent with previously obtained pairwise alignments. This invention is described in more detail below. It will be appreciated that this method may be used independently of the method of database generation described herein. This novel and inventive method described herein in the context of generating a relational sequence database is considered to form a separate invention and is the subject of a separate co-owned United Kingdom patent application.

According to this aspect of the invention, there is provided a computer-implemented method of aligning a plurality of protein or nucleic acid sequences comprising the steps of:

a) performing an alignment of a query sequence to a target sequence using a dynamic programming algorithm that constructs the alignment using a scoring matrix profile to provide an alignment score for aligning amino acid residues together, wherein suitable candidate residues for alignment are given a positive score and unsuitable candidate residues are given a negative score, and negative score penalties are generated both for opening and for extending a gap in one of the sequences in the alignment; and

b) repeating step a) for each sequence to be aligned;

wherein the scoring matrix profile is modified after each alignment step a) and before being used to generate the alignment of the next sequence, and wherein if the best scoring alignment requires that a gap be introduced into the profile, the profile is modified by inserting the residues from the query sequence that match up with the gap region.

In a similar way to known multiple alignment methods, the method of the invention uses a profile for the nominated sequence in an alignment strategy. The key novel concept behind the method of the invention is to allow the profile to be extended in regions where gaps are desired. Using pre-generated profiles as a basis for the multiple alignment permits this alternative strategy to be implemented. Preferably, a pairwise alignment strategy is used.

By “target sequence” is meant the nominated sequence on which the multiple alignment strategy is to be based. It is this sequence which is represented in the profile when the multiple alignment is commenced. This profile for this nominated target sequence is then aligned against a plurality of query sequences in turn, with the profile being modified by the alignment algorithm as the alignment proceeds.

In theory, any number of query sequences may be aligned against the profile for the target sequence. However, preferably, a selection of related sequences are used. Such a selection may be selected from the results of an iterative alignment program such as PSI-BLAST.

Preferably, the method of the invention is used to perform multiple alignments of protein sequences. Accordingly, the more detailed aspects of the invention that are described below refer to only to amino acid residues, in the context of aligning protein sequences.

However, the skilled reader will appreciate that the method of the invention is equally applicable to the alignment of nucleic acid molecules. Furthermore, it is envisaged that this method could easily be extended to allow the alignment of any string of letters where individual letter types have defined degrees of similarity. By “letter” is meant any character forming strings which it is desired to align together, and thus “letter” may include an ascii code.

In a preferred embodiment of the invention, the query sequences are aligned against the target sequence in order of their similarity to the target sequence. This degree of similarity may be assessed by degree of evolutionary divergence, for example, as defined by a similarity score generated by an alignment program such as PSI-BLAST. Preferably, a threshold similarity score is used to define the limit of similarity that a query sequence may display with a target sequence in order to be included in the multiple alignment method. This prevents the program that implements the process of the invention from attempting to align sequences that are too dissimilar to align to the target sequence. For example, for a sensible alignment to be generated, attempting to align a sequence that was not detected as being related to the target sequence by PSI-BLAST (and hence in this example the profile to be used in the alignment) would be inadvisable.

The basis of the novel algorithm that implements the method of the invention is the global alignment of two sequences using a dynamic programming algorithm, such as the pairwise alignment strategy described by Myers & Miller (Myers and Miller, Comput Appi Biosci (1988) 4(1):11). However, the novel method uses a profile-based scoring scheme when constructing the alignment. This is where the score for aligning two residues or nucleotides is not fixed globally, but varies with position along one of the sequences, this sequence always being the nominated sequence for which the multiple alignment will be constructed.

This profile is then used to generate the alignment with a target sequence. However, one or the key points for generating a multiple sequence alignment using this approach is to allow further modification of the profile. After each pairwise alignment is calculated, the profile is modified as shown in FIG. 2, as each of the sequences is aligned against it. Where the alignment calls for a gap in the profile, the profile is modified by inserting, from the aligned sequence, the residues or nucleotides that match up with the gap. These inserted residues or nucleotides are marked as such, as they have an effect on subsequent alignments of query sequences. The scoring values that these inserted residues are given may be taken from a standard scoring matrix such as any of the BLOSUM or point accepted mutation (PAM) series. A particularly suitable matrix has been found to be the widely used BLOSUM-62 matrix. Other suitable matrices will be clear to those of skill in the art.

After the pairwise alignment of each target sequence with the query sequence, the profile for the target sequence is modified before being used to produce the alignment for the next query sequence. Areas in the profile that have been modified are marked as such, as they affect the way that the alignment is scored in the dynamic programming step. This procedure is repeated for each sequence in turn until the complete alignment is produced.

In a preferred embodiment of the invention, if amino acid residues in a second or subsequent query sequence are aligned against a modified region of the profile where residues have been inserted and said amino acid residues are assigned a negative score, their score is reset to zero, such that multiple sequences that have similar regions that were not present in the original profile may be aligned together without penalty while at the same time allowing the alignment score to be increased for correctly aligned regions that have a positive score.

If the alignment of a second or subsequent query sequence requires that a gap be inserted or extended into the sequence that is being aligned against the profile and this gap falls within a modified region of the profile where residues have been inserted, no negative score penalty is generated. In this fashion, a sequence that would normally align against the profile without the need for a gap can be aligned without an inserted region interfering with the alignment.

The scoring matrix profile used in the alignment method may be a profile generated by running a profile-based alignment algorithm such as PSI-BLAST on the target sequence. However, a default scoring matrix may be used, if necessary. Suitable scoring matrices will be well known to those of skill in the art and include the BLOSUM and PAM matrices, particularly PAM 250 and BLOSUM 62. Preferably, the profile originates from running PSI-BLAST with the target sequence.

If a query sequence has previously been aligned by another method, and it has been discovered that the query sequence can align against the nominated target sequence in multiple locations, it is necessary to put this sequence through the algorithm multiple times, one for each of these ‘local hits’. The alignment produced for each appearance of the sequence must be constrained so that the correct local hit is chosen, rather than aligning the best area repeatedly. This constraint mechanism can also be used to make sure that particular areas of interest that have been previously identified are preserved by the alignment procedure.

Accordingly, this aspect of the method provides that if a query sequence is known to align against a target sequence in multiple locations such that multiple alignment hits are generated by the alignment of these sequences, then step a) is repeated for each location at which the sequences align, and for each separate iteration, the alignment of the sequences is constrained to one particular alignment location. This mechanism of constraint excludes regions from consideration by the dynamic programming algorithm by setting the matrix profile scores in the excluded region to a large negative value that is far more negative than any value that would occur naturally during the execution of the algorithm. Conveniently, this large negative value that is assigned is the largest negative value that can be stored by the computer on which the alignment method is being performed.

The effect of using a constraint mechanism as described above can be seen from FIG. 3. In this figure, the calculated alignment enters and exits the constrained region in the centre at the given points at either corner. However, within the central region, and the two other areas at either side, the alignment algorithm is free to proceed as normal. This means that it is possible approximately to specify a general area of interest and the alignment will find the best alignment within that region.

One advantage of this algorithm is that it can be performed in O(n) time, where a full multiple alignment requires O(n²) time. This means that the primary use of the method of the present invention is in interactive systems, where the alignments must be produced quickly in response to user requests. In such situations, it is expected that the sequences that are required to be aligned will have already been shown to have a reasonable degree of similarity, at least within certain regions, which is where this method performs best.

Only the profiles generated by the database search need to be stored in the database. Multiple alignments can be reconstructed from the stored profiles upon a user request.

One or more threading-based approaches are also used to analyse the sequences in the database. Many threading-based approaches are based on the seminal work of David Jones. His original approach to fold recognition is simple in concept and has proved to be highly effective. Firstly, a library of unique protein folds is derived from a database of protein structures and from these are derived use a set of statistically determined potentials. Each fold is considered as a chain tracing through space; the original sequence being ignored completely. The test sequence is then optimally fitted to each library fold (allowing for relative insertions and deletions in loop regions), with the ‘energy’ (score) of each possible fit (or threading) being calculated by summing the proposed pairwise interactions. The library of folds was then ranked in ascending order of total energy, with the lowest energy fold being taken as the most probable match.

There are now many suitable approaches, any of which could be used in the method of the present invention. In a particularly preferred embodiment of the invention, a method for fast genome threading is used that is particularly suitable for the vast numbers of sequences that need to be processed. The approach is an extension of the approach proposed recently, again by David Jones (Jones (1999) J. Mol. Biol., 287(4): 797-815). This approach preferably uses a traditional sequence alignment algorithm, a sequence and a profile to generate alignments which are then evaluated by a method derived from threading techniques. As a final step, each threaded model is evaluated by a neural network in order to produce a single measure of confidence in the proposed prediction.

In detail, the method starts by taking a representative set of known three-dimensional structures and calculating statistical potentials for the residues and interactions. In this method, the accessibility or solvation potential is considered for a given residue type. This is the area of a residue's side chain that is accessible to a solvent such as water. The second is the distance between atoms within pairs of residues, that also takes into account the linear separation of the residues along the protein chain, and the local secondary structure of the residue. This set of statistical potentials need be calculated only once, with the subsequent calculations making use of these pre-calculated values.

To apply the method, a sequence of unknown structure is aligned against sequences from proteins of known structure. This can be done using any alignment procedure. Preferably, however, both a local and local-global dynamic programming algorithm are used. The two sequences are then compared and a “profile” (mutation potential matrix) applied to one, to investigate areas of similarity between the two sequences. In a first, forward mode, the profile for the structured sequence is used to look for alignments with the other sequence. In the second, reverse mode, the profile for the unstructured sequence is used to look for alignments with the structured sequence. The alignment program generates a proposed alignment and a value representing the confidence of this alignment. Most preferably, the algorithms used are Smith-Waterman (for local alignments) and a method based on Myers-Miller's algorithm (for global alignments).

In the second step of the method, matches are made between a structure and a sequence of unknown structure, based upon the alignments generated in the first step of the threading method. When a sequence has been aligned against a known structure, the recalculated potentials for finding the residues from the query sequence in that conformation are then summed along its protein chain, to give total energies for both the solvation and pairwise interaction. These two potentials, along with the score from the alignment stage, are then passed through a neural network that has been trained on a set of known structures to give a single score value.

In order to aid interpretation, the results from a set of known structures which have been passed through the above procedure can be analysed to produce a mapping from the neural-network score to a confidence value. This expresses the results from the algorithm as the probability of the unknown sequence having the same structure as that of the structure to which it was compared.

The results of the threading-based data analysis are then loaded into the database.

According to a further aspect of the present invention, there is provided a database generated by a method according to any one of the aspects of the invention that are described above.

The database of the invention may be utilised in conjunction with a user-controlled computer-implemented prediction program to predict the function of a protein sequence for which no functional information is known. The user inputs a query protein sequence into a prediction program, which then interrogates the database to assess the degree to which the query sequence matches sequences for which alignment data have been pre-calculated. Based on these data and the degree of matching with other sequences and structures, predictions are made of the biological function of the query protein sequence. Because of the huge number (over 100,000) of interrelationships that were used to test the approach, the confidence that can be placed in predictions made using the programs of the invention is extremely high.

Accordingly, a further aspect of the present invention provides a computer apparatus adapted to compile a relational database using a method according to any one of the aspects of the invention described above.

The computer apparatus may comprise the following elements at least: a processor means; a memory means adapted for storing data relating to amino acid sequences and the relationships shared between different protein sequences; first computer software stored in said computer memory adapted to align said protein sequences using one or more pairwise alignment approaches; second computer software stored in said computer memory adapted to align said protein sequences using one or more profile-based approaches; and third computer software stored in said computer memory adapted to align said protein sequences using one or more threading-based approaches.

The memory means may be adapted for storing data relating to:

(a) the sequences of a plurality of proteins;

(b) the structures of a plurality of proteins;

(c) the predicted alignments of each of said sequences with every other one of said sequences;

(d) the predicted alignments of sequences of known structure with those of unknown structure.

In an alternative aspect, the computer apparatus may comprise the following elements:

a processor means;

a computer memory for storing data;

first computer software stored in said computer for comparing a specific sequence of amino acid residues to amino acid sequences stored in a database as described in the above aspects of the invention;

second computer software stored in said computer for presenting the results of said comparison step in an application programming interface;

display means, connected to said processor, for visually displaying to a user on command a list of proteins with which said specific sequence of amino acid residues is predicted to share a biological function.

A still further aspect of the invention provides a computer system for compiling a database containing information relating to the interrelationships between different protein and/or nucleic acid sequences, said system performing the steps of:

a) integrating data from one or more separate sequence data resources into a combined database;

b) comparing each query sequence in the combined database with the other sequences represented in the combined database to identify homologous proteins or nucleic acid sequences;

c) compiling the results of the comparisons generated in step b) into a database; and

d) annotating the sequences in the database.

A still further aspect of the invention provides a computer system for compiling a database containing information relating to the interrelationships between different protein sequences, said system performing the steps of:

a) combining protein sequence data from one or more separate sequence data resources and one or more structural data resources into a database;

b) comparing each query protein sequence in the database with the other protein sequences represented in the database to identify homologous proteins using, for each query sequence:

i. one or more pairwise sequence alignment searches,

ii. one or more profile-based sequence alignment searches;

iii. one or more threading-based approaches;

c) compiling the results of the comparisons generated in step b) into a relational database; and

d) annotating the sequences in the database.

The invention also provides a computer-based system for predicting the biological function of a protein comprising the steps of:

a) inputting a query sequence of amino acids whose function is to be predicted into a database generated according to a method as described in any one of the aspects of the invention described above;

b) interrogating said database for sequences that are similar to said query sequence; and

c) presenting said related sequences in order of similarity with the query sequence, wherein the functions of the related sequences correspond to the functions predicted for the query sequence.

The computer-based system may be designed to enable the steps of:

a) accessing a database according to any one of the aspects of the invention described above;

b) inputting a query sequence of amino acids whose function is to be predicted into said database;

c) interrogating said database for sequences that are similar to said query sequence, and

d) presenting said related sequences in order of similarity with the query sequence, wherein the functions of the related sequences correspond to the functions predicted for the query sequence.

The database may be located at a site remote from the user computer, such as for example, an Internet server.

Such a computer system may comprise the following elements:

a central processing unit;

an input device for inputting requests;

an output device;

a memory;

at least one bus connecting the central processing unit, the memory, the input device and the output device;

the memory storing a module that is configured so that upon receiving a request to predict the biological function of a protein, it performs the steps listed in any one of the methods of the invention described above.

In a particularly preferred embodiment of the invention, a user interface may be provided that facilitates access to the relational database of the above-described aspects of the invention. The user interface may be loaded onto any processor-based system, either general purpose or special purpose. The term general purpose is meant to include any processor-based system such as a personal computer, a portable processor such as a personal digital assistant, a part of a network, a server and so on. Special purpose systems are processors set up for the specific purpose of providing access to the relational database and viewing results of user queries.

The user interface may be linked directly to the relational database, or may be linked via a local or remote network linkage, for example, via the Internet. In the latter embodiment of this aspect of the invention, access to the database should preferably be via a secure link, limiting access to the database to users that input a specific password or are required to perform part of any other secure handshaking procedure.

The design of the user interface allows a user to access the contents of the relational database, either by way of a user-defined input query, or simply by browsing the database entries, as required. The interface should be loaded with one or more tools for the visualisation of sequence alignment, three dimensional protein structure and protein-ligand relationships. In a preferred embodiment, the interface is loaded with a computer program that allows a user to view alignments of protein sequences contained within the relational database, a viewer program capable of displaying three-dimensional structures of sequences in the database, and a second viewer program allowing the display of interactions (real or predicted) between protein structures and ligand molecules.

In a particularly preferred embodiment of this aspect of the invention, specially enhanced versions of leading visualisation tools are used, including an interactive editor for multiple sequence alignment, such as the program termed “AlEye”. Any other similar tool could be used if required, such as, for example, the CINEMA program that is currently used widely. A protein structure viewer, such as RASMOL, the industry-standard 3D protein structure viewer, or an enhanced version of this program may also be used. A program developed by the inventors, termed “LigEye”, which is an interactive tool for viewing protein-ligand interaction diagrams generated by the LIGPLOT tool (Wallace et al., (1995) Prot. Eng. 8: 127-134) may also be used. However, any other program that performs the same or similar tasks also be used. These tools should work in an integrated fashion to provide co-ordinated visual information on the proteins under study at the sequence, structural and functional levels.

An alignment editor is a visual tool that allows multiple sequence alignments to be viewed and adjusted relative to one another. The ability not only to view but also to edit alignments is a critical tool in sequence analysis, since automatically calculated alignments may require manual adjustment to remove spurious gaps, restore residue windows or otherwise correct misalignments. Preferably, the AlEye alignment program is used, as described herein.

AlEye is written in the Java language. It allows the viewing of pre-generated sequence alignments as well as the generation of sequence alignments by hand. Alignments are edited by clicking on the sequences and dragging them to create gaps; whole sequences may be shifted to the left or right by clicking on the right mouse button and dragging. In the present implementation, the program shows secondary structure and hydrogen bond information, although any information from the database that refers to specific residue positions (for example, PROSITE regular expressions, hydrophilic structure interactions) could be used.

Preferably, the alignment is coloured according to residue type, although other schemes could of course be used, such as secondary structure. if known, regular expressions and protein-ligand interaction data. Since proline and glycine have special structural properties, particularly in membrane proteins, they are grouped separately, and an additional category is provided for cysteine, which is often involved in disulphide bond formation. The user is able to select between various alternative colour schemes or to modify the background colours for each amino acid on a one-to-one basis.

Visualisation of protein structure in three dimensions is extremely helpful in understanding protein function and for the analysis of drug targets. In order to view three dimensional protein structures, a number of viewing programs are available. An example includes the publicly-available molecular graphics program RASMOL (available at http://www.umass.edu/microbio/rasmol/getras.htm). This program allows a molecule to be viewed in three dimensions in a variety of formats and, through movement and rotation of the image, to be viewed from any chosen perspective.

The RASMOL program reads in molecular co-ordinate files and interactively displays the molecule on the screen in a variety of representations and colour schemes. The loaded molecule can be shown as wireframe bonds, cylinder ‘Dreiding’ stick bonds, alpha-carbon trace, space-filling (CPK) spheres, macromolecular ribbons (either smooth shaded solid ribbons or parallel strands), hydrogen bonding and dot surface representations. Different parts of the molecule may be represented and coloured independently of the rest of the molecule or displayed in several representations simultaneously.

Importantly, the displayed molecule may also be rotated, translated, zoomed and z-clipped (slabbed) interactively using either a mouse, the scroll bars, the command line or an attached dial box. This is of great utility in understanding the three-dimensional structure of a protein since it permits the user to move continuously around the molecule to any chosen perspective.

In a preferred embodiment of the invention, the interface for use in the present invention uses an enhanced version of this program. This may include the following additional features not present in the standard RASMOL program.

The analysis of protein-ligand interactions plays an important role in drug design since many drugs act by preventing or mimicking such interactions. Protein-ligand interactions are mediated by hydrogen bonds and hydrophobic contacts, but the exact nature of such non-covalent interactions are extremely difficult to visualise in three dimensions.

Any computer-implemented method of visualising protein ligand interactions may be used in the interface. In a preferred embodiment of this aspect of the invention, visualisation of protein-ligand interactions may be achieved using the LigEye visualisation program that enables protein interactions to be viewed in two dimensions. The LigEye program may be fully integrated with the advanced RASMOL program so that the either the full or a highlighted part of the three-dimensional structure can be viewed simultaneously with the two-dimensional LigEye representation. The integration of RASMOL and LigEye is a powerful facility that significantly increases the functionality of the relational database in target analysis.

LigEye is a viewer for diagrams generated by LIGPLOT (Wallace et al., (1995) Prot. Eng. 8: 127-134), a program that automatically generates clear, two-dimensional representations of such interactions. These diagrams are particularly useful for illustrating the interaction between different ligands (for example, two different drug candidates) and the same target enzyme, or for comparing different enzymes.

The LIGPLOT program automatically generates schematic diagrams of protein-ligand interactions. The algorithm reads in the 3D structure of the ligand as specified in data parsed from the Protein Data Base, together with the protein residues it interacts with, and ‘unrolls’ each object about its rotatable bonds, flattening them out onto the 2D page. Thus, the LIGPLOT program collapses the three-dimensional structure of the protein and ligand into two dimensions. Preferably, all the atoms of the ligand are represented on the plot, and the ligand atoms can also be colour coded to indicate their accessibility to the solvent. However, the full structure of the protein is not illustrated. The following information is available:

Only those amino acid side chains within the protein that are hydrogen bonded to the ligand are shown in full, with an option to include or exclude its main chain atoms. Hydrogen bonds between the protein and the ligand are illustrated by dashed lines between the atoms involved.

Hydrophobic interactions are shown more schematically. Residues from the protein that are involved in these interactions are depicted as an arc with spokes radiating out towards the ligand they contact. The contacted atoms are shown with spokes radiating back.

This program will work for any ligand and will provide a clear schematic representation of the types and locations of the ligand's non-covalent interactions. In the relational database, Ligplots can be edited by the user for increased clarity, and cross-referenced with three-dimensional representations generated by the RASMOL program.

The stages of the LIGPLOT algorithm are as follows:

Stage 1. Identification of co-ordinates. The three-dimensional co-ordinates of the protein and ligand are read in from the protein structure data (Protein Data Base data) and the atoms involved in hydrogen bonded or hydrophobic interactions are identified, using the program HB discussed above (Baker and Hubbard, op. cit.).

The program computes all possible positions for hydrogen atoms attached to donor atoms which satisfy specified geometrical criteria. LIGPLOT also has an option that allows additional side chains not directly bonded to the ligand to be included. This allows more distant hydrogen bonds to be included, as well as hydrogen bonds between the protein and ligand that are mediated by one or more water molecules.

For hydrophobic groups, the specific side chains involved are not shown and a single position is used for the residue as a whole. This position is linked to the atoms on the ligand with which it is in contact by ‘virtual’ bonds. This simplifies the unrolling procedure while making the final representation more informative.

The covalent connectivity of the remaining atoms is then calculated, and certain bonds are cut to facilitate the unrolling procedure. For instance, if two adjacent amino acids are both hydrogen bonded to the ligand, the peptide bond joining them will be removed so that they can be moved independently when the structure is unrolled and cleaned up.

Stage 2. Identification of bonds for rotation. The unrolling procedure used in LIGPLOT depends upon rotatable bonds, i.e. bonds in which the structures to either side can be rotated or otherwise moved independently of the structures on the other side of the bond. For instance, bonds that are part of a ring are non-rotatable, since moving the structure on one side of them affects the structure on the other side by virtue of the ring connection.

This applies not just to recognised rings but also to any closed loop structure within the molecule. Closed loops are in fact quite common when hydrogen bonds are taken into account, and to overcome this problem one of the hydrogen bonds is made ‘elastic’ (i.e. capable of being stretched and distorted) while the other is treated as a rotatable bond.

Ring groups are flattened at this stage to ensure they are perfectly planar before the unrolling procedure takes place.

Stage 3. Unrolling the structure. The unrolling of the structure is the crux of the LIGPLOT program. To either side of each rotatable bond, the structure is rotated so that the bonds springing directly from its two ends come to lie in the same plane. Repetition of the procedure on all rotatable bonds in turn gives a structure that has been completely flattened into a single plane. The unrolling procedure is carried through working from one end of the ligand to the other, although where branching occurs the branches have to be unrolled in turn. None of the bond lengths are disturbed in the unrolling process, and some of the bond angles are maintained.

Stage 4. Clean-up. Although completely flat, the structure at this stage will probably include extensive overlap between atoms and bonds, resulting in a very congested and confusing diagram of interactions. A clean up procedure addresses this problem. Once again, each rotatable bond is cycled through in turn, with a test made for each bond to see if a rotation of the structure through 1800 on one side of the bond will reduce the number of atom clashes and bond overlaps. The severity of the overlaps is evaluated using a simple energy function combining the energy due to close contact of non-bonding atoms and the energy due to bond overlaps. The entire cycle of all possible 180° flips is repeated several times until the number of atom and bond overlaps reaches a minimum.

Stage 5. Plotting. Once the clean-up procedure has been completed, the final structure is plotted. Plotting can be carried out in colour or black-and-white, the colours of atoms and bonds can be defined by the user, molecules can be shown as bonds only or in ball-and-stick form. A range of other user-defined viewing options are available. Once the plot has been produced, the user can modify the positions of the residues surrounding the ligand to enhance the clarity or realism of the image.

The additional features of the LigEye program include features such as an ability to rearrange the positions of the interacting residues by translation and rotation, and inclusion/exclusion of specific hydrogen bonding information by drawing lines between interacting atoms/residues.

Together, the programs that form part of the interface should provide the user with ways to view information on individual proteins, or to highlight the relationships that link a group of proteins together. This provides a user with a wide range of options for filtering the data that may be accessed from the relational database so that a user can focus on proteins that are most relevant to his/her work. Preferably, a windows-based approach is used for the interface such that each interface program appears on a display screen as a separate window.

Preferably, the relational database is installed on a server machine, thus allowing many individuals to share a centralised source of data. However, the Interface programs should generally be installed on individual desktop machines for all those individuals who require access to the relational database.

As one example of how the features of such an interface program might be designed, a program termed “Workbench” will be briefly described below. The skilled reader will appreciate that once the general concepts outlined below are understood, similar interface programs may be designed that share the advantageous features of Workbench.

Workbench preferably provides several possible entry points into the relational database by allowing the user to search for the proteins starting with a variety of different types of information.

For example, in one feature of Workbench, the user may compose a query that targets types of protein of interest. Workbench passes the query to the relational database server, which scans its stored information for proteins that match the search criteria used. If matching protein sequences are found in the database, their entry records are returned to Workbench, which lists them. At this point, the user might continue the analysis by working with Workbench, or alternatively might choose a selection of the listed proteins and view alignments of these selected sequences with other protein sequences predicted as being related by the relational database. When using AlEye, for each protein sequence case, the program indicates in the display page whether ligand or structural information is available for any of the loaded sequences. If any such additional information is available, this can be viewed using a viewer program, such as RASMOL (3 dimensional structures) and/or LigEye (predicted interactions between protein structures and ligand molecules).

In another example, the name of a completely sequenced organism may be selected and statistics displayed to give information about the genome of such an organism. Information can be given relating to the primary sources from which the information came (GenBank, SWISS-PROT or PDB), and what proportion of the sequences in the genome have direct, close and distant homologues as defined by secondary database relationships calculated within the relational database. Functional information, information relating to predicted secondary structures and details of kingdom classification can also be given.

A word search such using a concept or key word may also be used to search the relational database. By doing this, a group of proteins is selected by searching for specific words or phrases that are represented in annotations of these protein records in the relational database. Depending on the user's choice of search term, a search can be made for a relatively broad range of proteins, or for a few defined sequences.

Conveniently, search terms may search key words, entry descriptions (annotations in SWISS-PROT and PDB records), product descriptions (searches the text in the protein name and alternative protein name lines of GenBank records), functional descriptions (GenBank records), EC numbers (GenBank, SWISS-PROT, PDB), gene names, additional notations (the CDS note line of GenBank records), organism name, taxonomy ID, entry ID (the entry identified allocated to SWISS-PROT, GenBank and PDB records), authors, journal, title, date and so on. In line with conventional search engines, queries can be combined and refined, as necessary. The scope of these searches should ideally be controllable using logical operators and wild cards in the query terms used. Ideally, the query definition can be refined if too many sequences are returned by the initial query. A specific sequence of amino acids or nucleotides may also be input into the workbench interface. This allows a user to search for proteins that match a known sequence of amino acids or DNA nucleotides. Such a query may generate an exact result with one or more known sequences. Preferably, links may be provided from such a page to one or more other windows, showing for each sequence, other protein sequences that are predicted to align with the query sequence. Calculated relationships between the query sequence and all other selected sequences in the database, may also be shown.

The accession code or unique identifier of a database record may also be used as a search code. In this way, the workbench interface can provide a direct method for viewing information relating to a particular protein when the unique code is known by which it is identified in GenBank, SWISS-PROT, or PDB. Again, an extracted sequence list page may be used to allow cross-references to predicted alignments between the chosen protein and other proteins in the relational database.

The identity of a non-peptide ligand that may be associated with a protein of known structure may also be used as a query term. This provides a way to search the relational database for protein structure records (PDB records) in which the protein is reported in a complex with a known non-peptide ligand. If the workbench interface program finds proteins that match a submitted query, results can again be shown with cross-links provided to alignment pages and calculated relationships pages.

The residue sequence of a peptide ligand associated with a protein may also be used as a query term. This allows the searching of protein structure records (PDB records) where either a protein is determined to be in complex with a specific peptide ligand, or, following protein digestion, a short protein fragment interacts with the remainder of the protein. Again, the search results page may include cross-references to alignment pages and calculated relationships pages.

In order to view predicted protein relationships, the workbench interface allows a user to identify and investigate sequences that belong to the same non-redundant sequence family. Preferably, for each protein, a display page is provided that lists all the members of the sequence family and that provides links to their primary database record. For each family, links may also be included to a page that shows predicted alignments of other sequences against that record sequence, that identifies any mapping to secondary database motifs, and that provides links to the relevant secondary database records.

The workbench interface program also enables the user to focus on a limited number of potentially interesting sequences. For example, it might be desirable to look for a possible evolutionary relationship between one of these sequences and all the other sequences selected from the relational database. This type of analysis is supported by the database by virtue of the pre-calculated relationship data provided within it. Accordingly, an aligned sequence display page may be provided for each sequence, showing relationship data for associated sequences. Preferably, the results page displayed shows details such as clustering of sequences that are more than 90% identical and which are of a similar length, an alignment score for the calculated threading relationship, and a confidence value that assesses the predicted value of each score by assigning it a confidence value. However, other values could be equally applicable.

The invention will now be described by way of example, with specific reference to a database in which information from the primary databases GenBank, SWISS-PROT and the PDB has been collated and cross-referenced.

### BRIEF DESCRIPTION OF THE FIGURES

FIG. 1 shows a graphical representation of the region of alignment between two related sequences.

FIG. 2 shows the situation when the two alignment regions are disjoint.

FIG. 3 shows the situation when one region of alignment is completely enclosed by another.

FIG. 4 shows the situation when two regions of alignment intersect.

FIG. 5 shows a profile modified by the novel method of multiple alignment described herein.

FIG. 6 diagrammatically represents constraining an alignment.

FIGS. 7-20 are diagrams setting out the structure of the system specification for the database generation.

### EXAMPLES

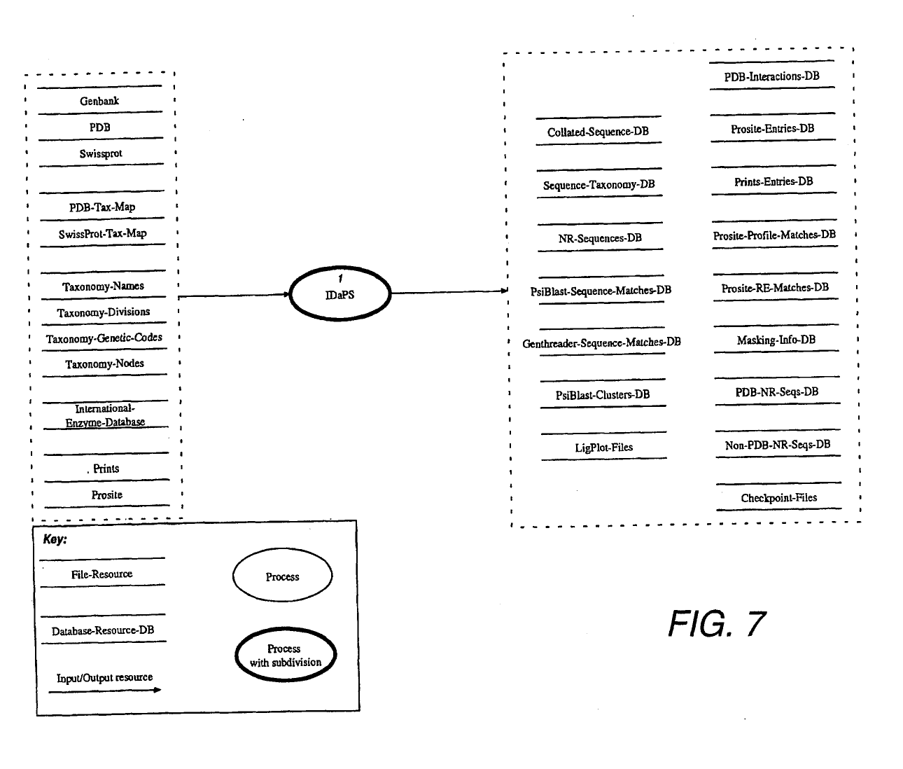

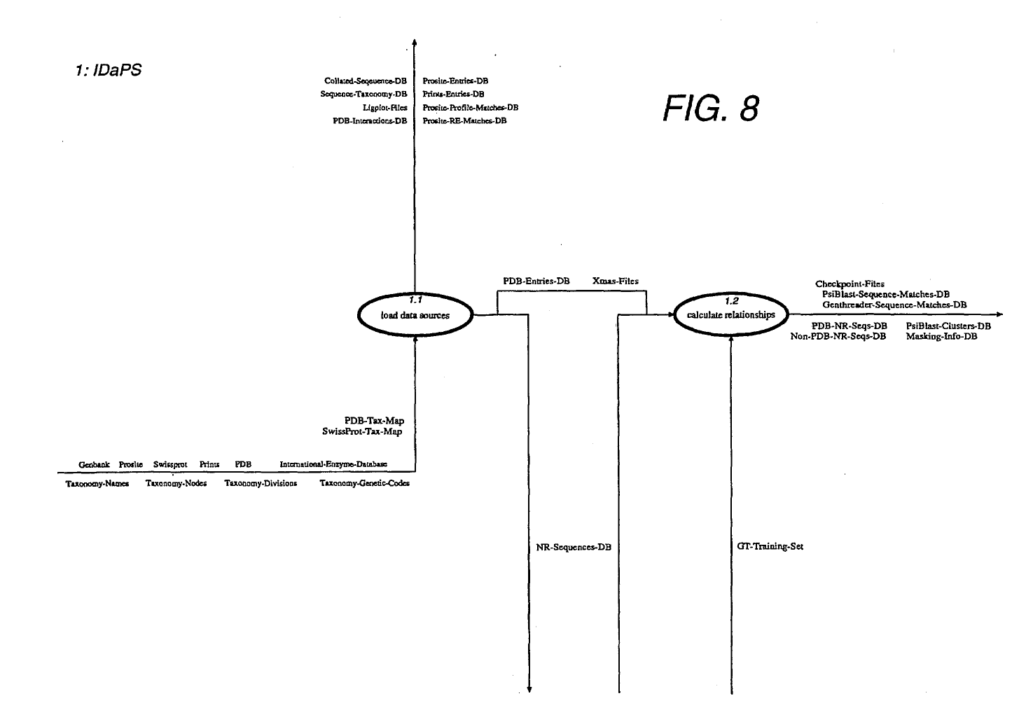

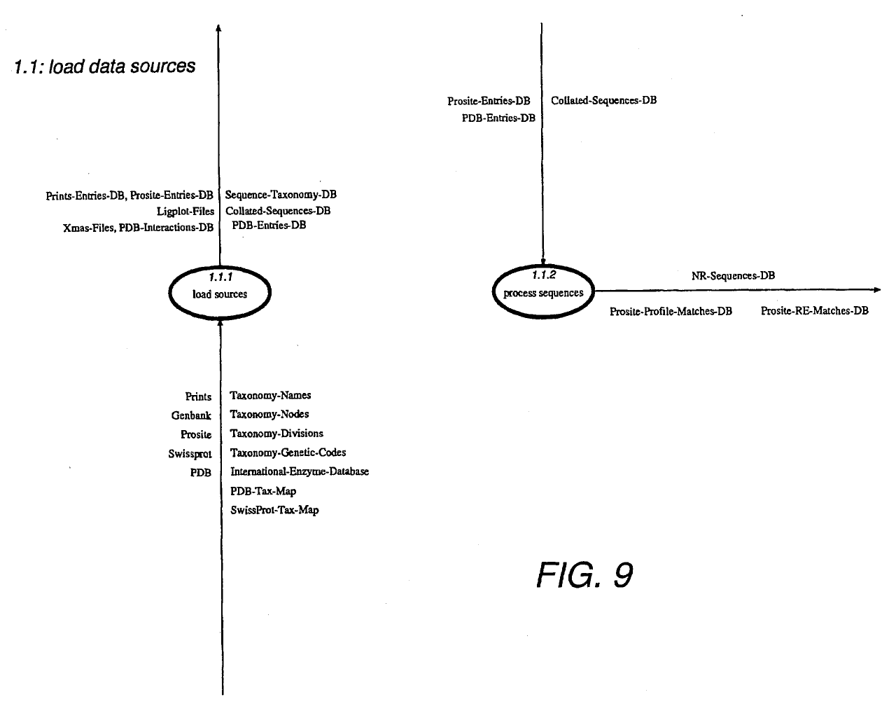

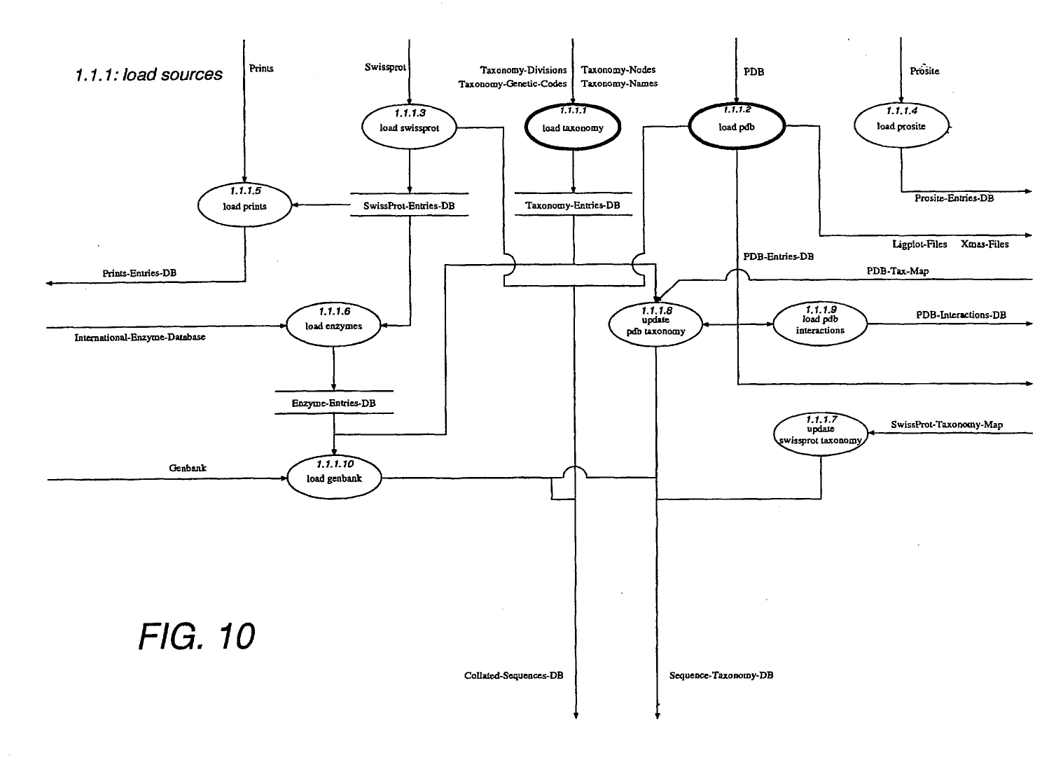

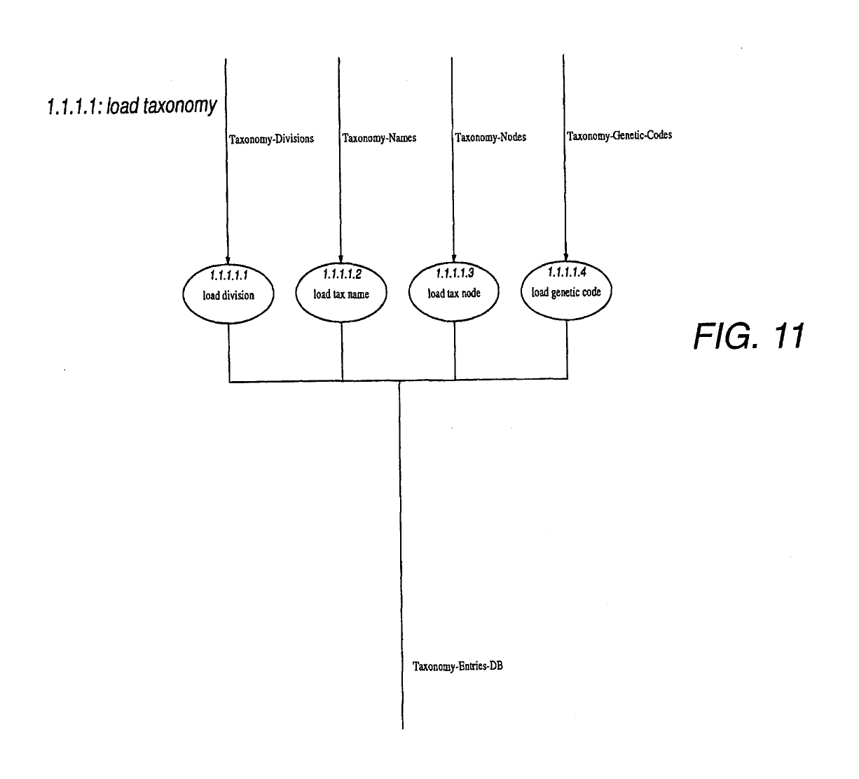

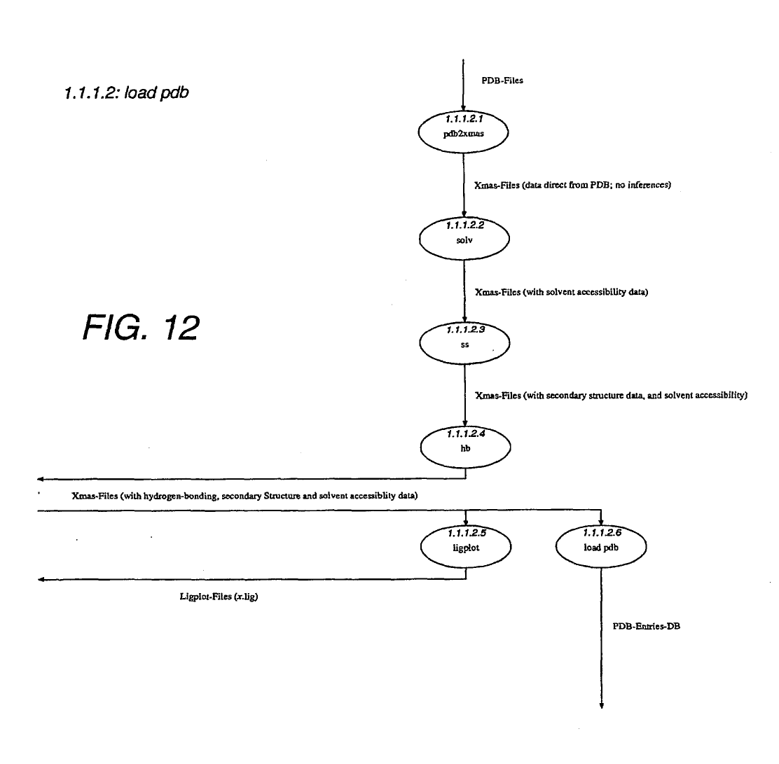

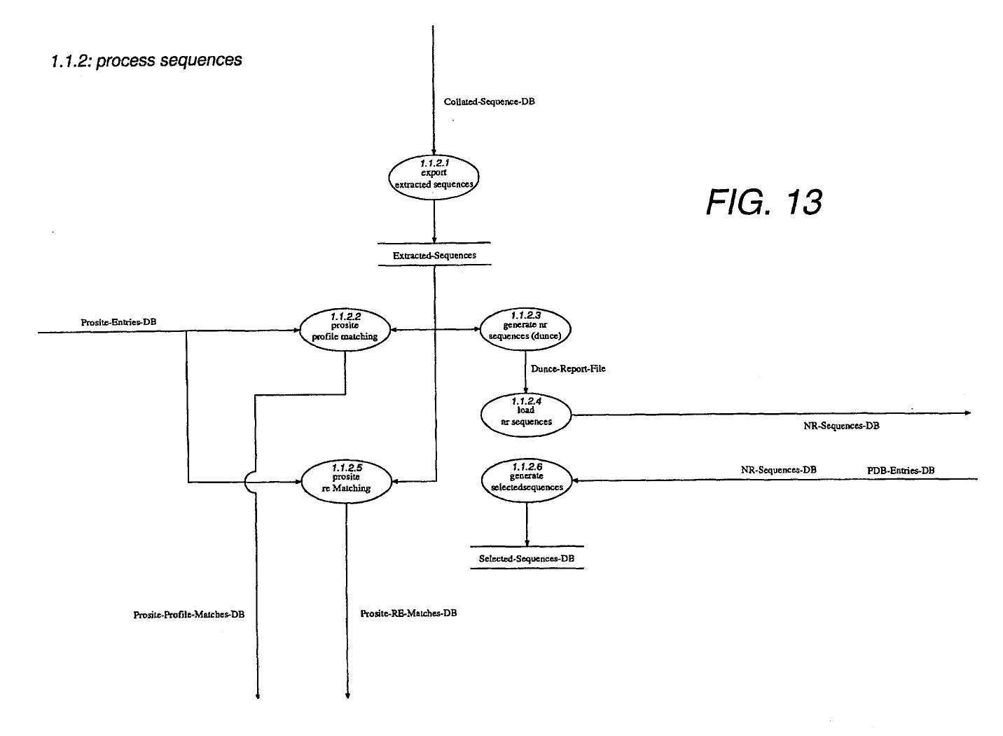

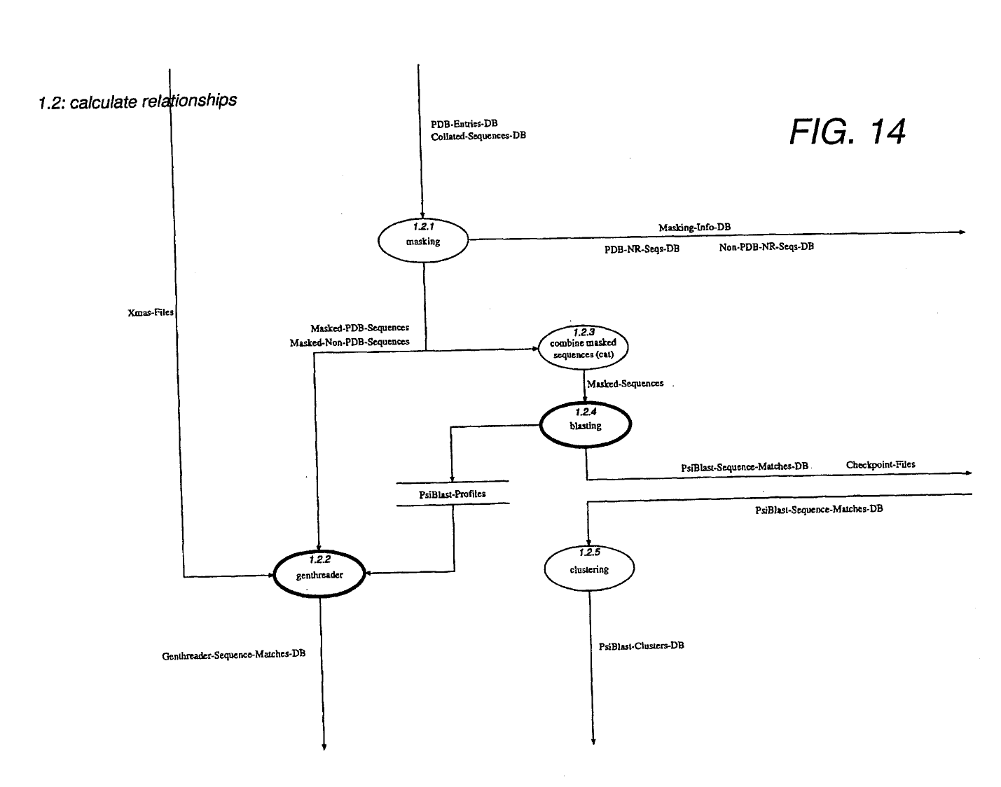

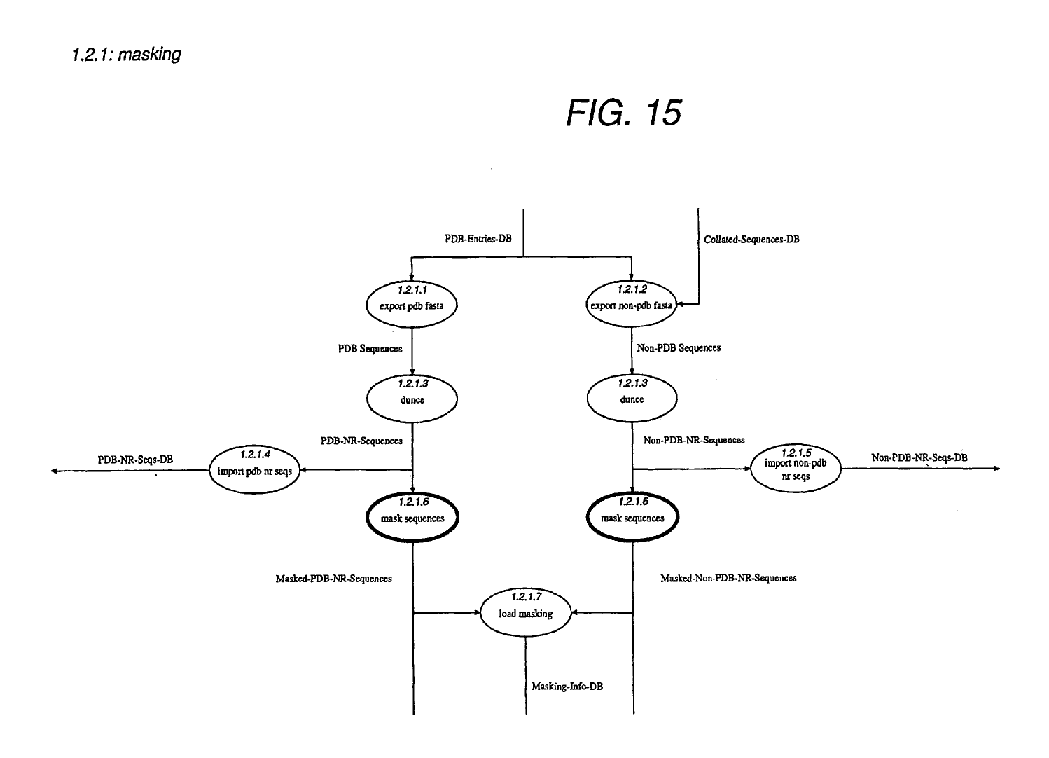

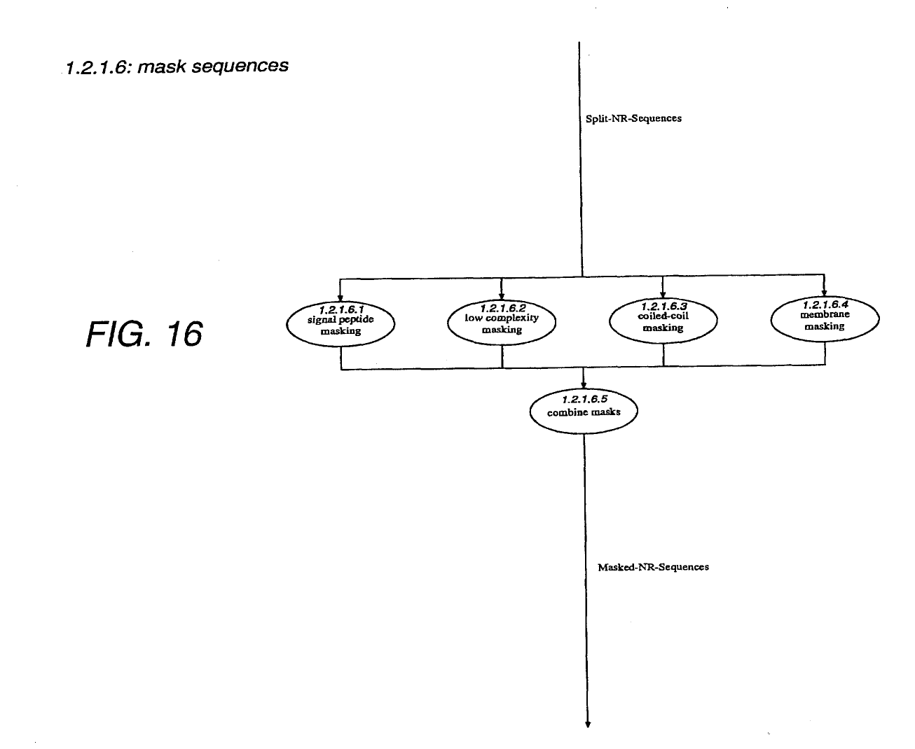

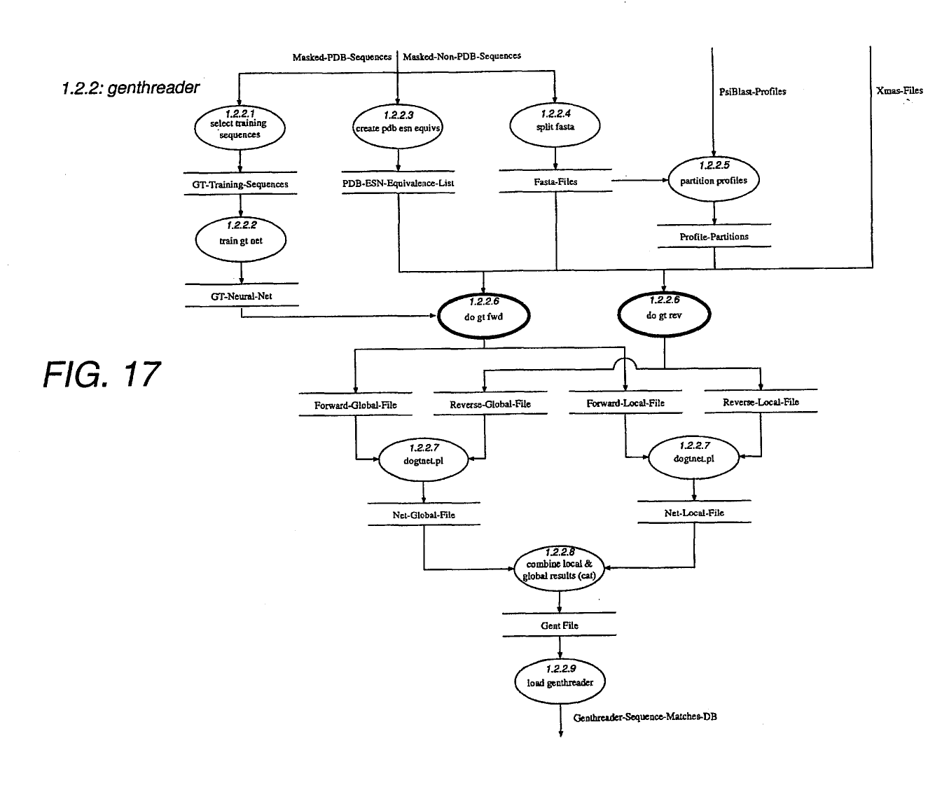

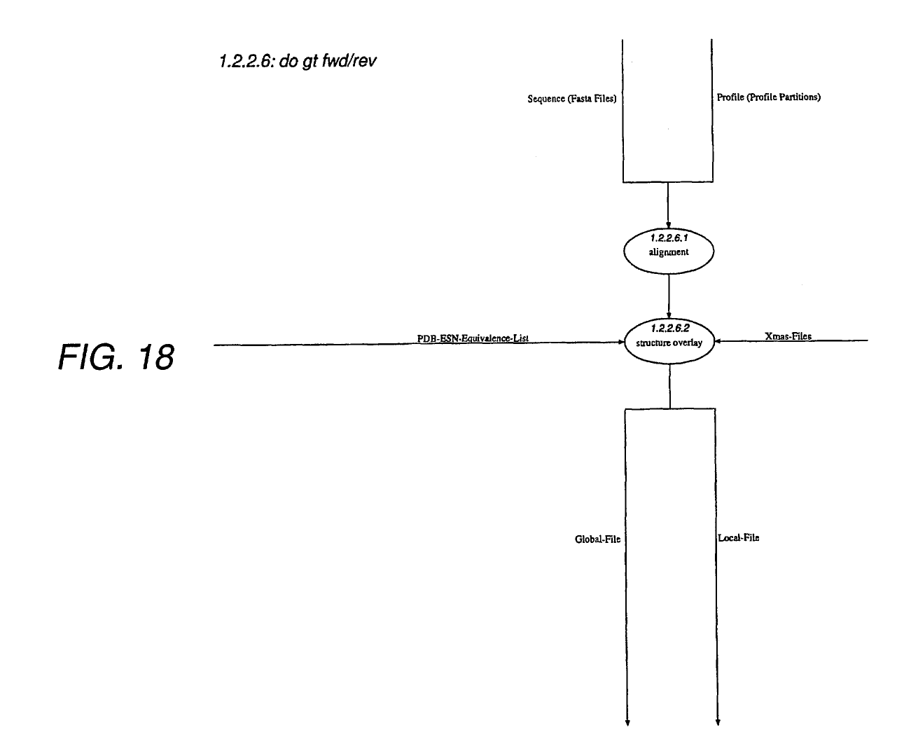

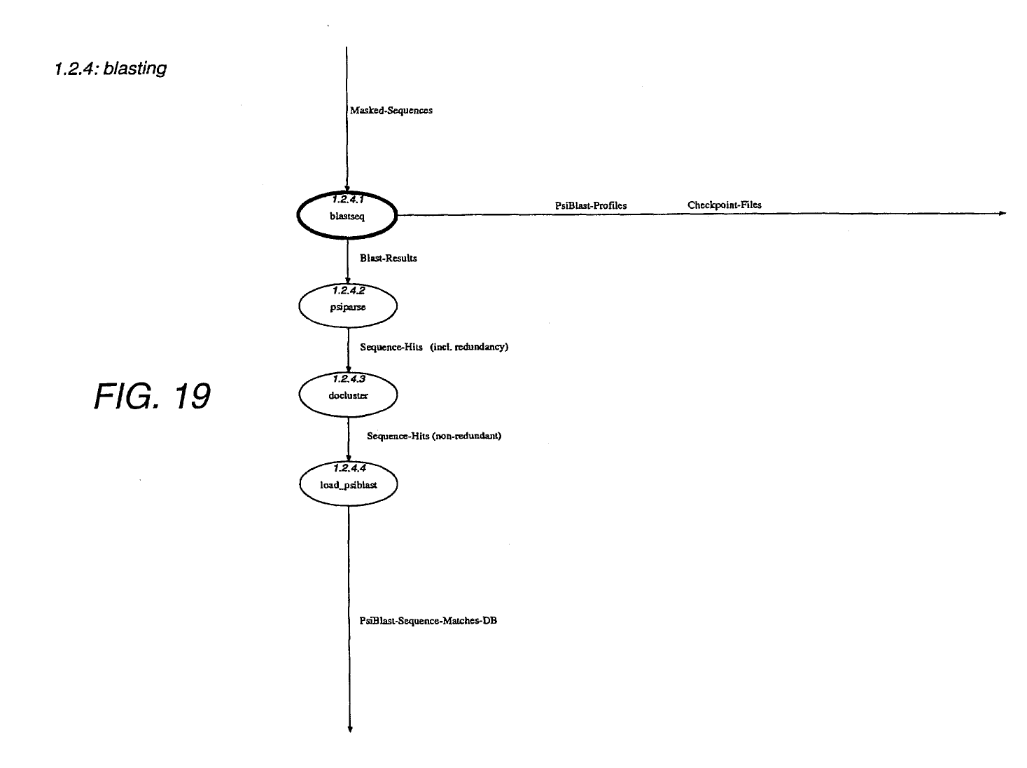

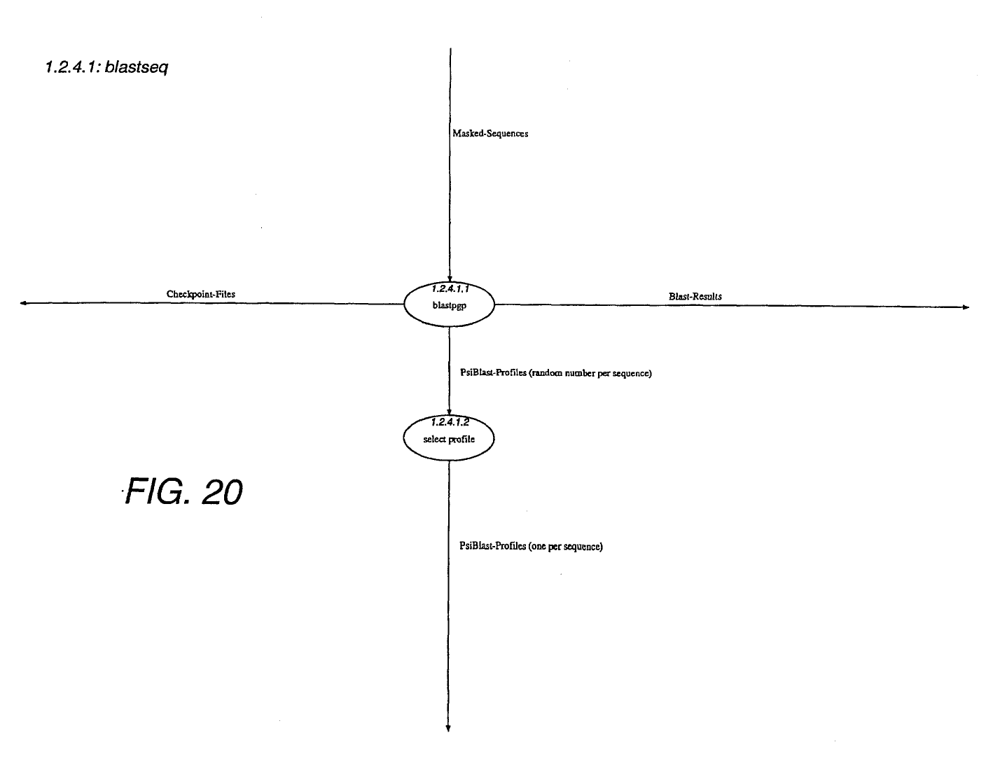

In the Examples below, the paragraph numbers correspond to the entries given for the same actions in FIGS. 7-20.

1. System Specification

Generate database of sequence relationships, collated sequence data, sequence selection data and database correlations.

1.1. Load Data Sources

Load and cross-reference public domain databases, select sequences of interest for comparison. Information is loaded from the primary databases GenBank, SWISS-PROT and the PDB, from the secondary databases PRINTS and PROSITE and additionally from the public domain databases Taxonomy (NCBI) and the International Enzyme Database.

1.1.1. Load Sources

Load public domain databases. Cross-reference sequence databases with taxonomy databases, Convert PDB files into internal formats for later use (xmas, ligplot).

1.1.1.1. Load Taxonomy

Load entries from the NCBI taxonomy database into the combined database (herein referred to as the CARSS [Combined Archive of Related Sequences & Structures] database).

1.1.1.1.1. Load Division

1.1.1.1.2. Load Genetic Code

1.1.1.1.3. Load Tax Node

1.1.1.1.4. Load Tax Name

1.1.1.2. Load PDB

Process PDB Files to produce xmas files, and ligplot files. Load PDB information from xmas files into database.

1.1.1.2.1. pdb2xmas

Convert pdb files into xmas format.

This program is believed to be able to parse all PDB files successfully (including Level 1 releases) or at least to identify errors in the files and mark them for manual correction.

Note that the residue number is read as a 5 character string to include the insertion code. The atom name, residue name and residue number references are padded with full stops to represent spaces. The program performs a fatal exit if memory allocation failed for any of these, if no atoms are read or if an error was identified.

1.1.1.2.1.1. Basic File Parsing

The following records of a PDB file are read by the parsing program. The action codes are explained below:

| | |
|---|---|
| ATOM/HETATM | ParseAtom () |
| TITLE/COMPND/HEADER | ParseHeader() |
| SOURCE | ParseSource() |
| AUTHOR | ParseAuthor() |
| DBREF/REMARK 999 | ParseSwiss() |
| CRYST1 | ParseCryst() |
| SEQRES | ParseSeqres() |
| CONECT | ParseConect() |
| REMARK 1 | ParseRemark1 () |
| JRNL | ParseJournal() |
| KEYWD | ParseKeywords() |
| REMARK 7 KEYWD: | ParseRemark7Keywords() |
| HET | ParseHet() |
| HETNAM | ParseHetnam() |

In addition, the program reads experimental information (resolution, R-factor, free-R, experiment type) using up to 5 passes through the headers. The experiment type is one of: XRAY, NMR, MODEL, UNKNOWN. For numeric fields which cannot be set, the marker value of 0.0 is used.

The following routines parse basic information:

ParseHeader( ) :If there is a TITLE record, the title is inserted. From the HEADER record, the date and PDB code are taken. All COMPND records are appended to obtain compound information and a truncated version of this is used for the title if there is no TITLE record.

ParseSource( ): All SOURCE records are appended.

ParseAuthor( ): All AUTHOR records are appended.

ParseCryst( ): the unit cell parameters and space group are parsed out.

ParseSeqres( ): the sequence is extracted from the SEQRES records. For each chain, the chain type is set to protein or DNA. The original 3-letter residue names from SEQRES is stored as well as the 1-letter version. This element of the program also warns if the number of residues specified within the SEQRES records is less than the number of residues read.

ParseSwiss( ): the SWISS-PROT database links are read from the DBREF records. REMARK 999 records and crosslinks to databases other than SWISS-PROT are not currently recorded.

ParseAtom( ): the ATOM and HETATM records are read, stopping at the end of the first MODEL. The base atom types are set as ATOM or HETATM depending whether this was an ATOM or HETATM record. The residue number and any associated insertion code are read into a 5-character string.

ParseConect( ): Reads in the CONECT records. Only the 4 potential covalent bonds are read.

ParseRemark( ): Concatenates REMARK1 records onto the references string.

ParseJournal( ): Concatenates JRNL records onto the journal string.

ParseKeywords( ): Parses the KEYWD records—on output, the keyword information is split at each comma.

ParseRemark7Keywords( ): Parses the “REMARK 7 KEYWD:” records (as seen in 1lmk) —on output, the keyword information is split at each comma.

ParseHet( ): Reads the HET records which form a dictionary of what the HETATM residues consist of. The residue name, the chain and residue number and the text description (which may be blank) are read. Any textual data from HETNAM records is used to replace this text.

ParseHetnam( ): Reads the HETNAM records which form a dictionary of what the HETATM residues consist of. The residue name and the text description are read. Before writing the XMAS file, data from HETNAM records is merged with the HET data. It replaces any text descriptions from associated HET records (Performed by FixupHetNames( )).

1.1.1.2.1.2. Simple Atom Cleanup

Simple Atom Cleanup is performed as follows.

Alternate occupancies are removed (RemoveAlternate( )), keeping only the highest occupancy or the first if there are more than one the same. If an alternate is found, then it is stored while the other ones are searched for. First the current residue is investigated. This will work for the vast majority of files where the alternates are with the main atoms. If the alternates are not found within the residue, then the rest of the records are searched. This covers at least some of the known entries where the alternates are placed at the end of the file. However, some will still not be found by this procedure (those cases where the alternative field in the PDB file has not been correctly filled in) and will record an error later (due to two identical residue identifiers).

Of the alternates identified, that with the highest occupancy is selected, defaulting to the first if the occupancies are the same.

By default, pseudo atoms are removed. If the experiment type is NMR, then atoms with the first two characters as “.Q” are removed. (StripPseudoAtoms( )).

By default, hydrogen atoms are removed. Atoms with element type “H.” or “D.” are removed. (StripHydrogen( )). Note that element types must first be assigned by SetAtomElement( ).

1.1.1.2.1.3. Setting Atom Types

Each atom is assigned one of the following types: ATOM, NUC, MODPROT, MODNUC, NONSTDAA, NONSTDNUC, NTER_ATTACHMENT, HETATM, METAL, WATER, BOUNDHET.

SetSimpleAtomTypes( ) is used to set the atom type field for waters and nucleotides. If the base atom type (i.e. as seen in the PDB file) is HETATM, then the residue names HOH, OH2, OHH, DOD, OD2, ODD, WAT are searched for in order to change the atom type to WATER.

If the base type is ATOM, then we look for residue names “..A”, “..C”, “..G”, “..I”, “..T”, “..Y”, “..U”, “.+A”, “.+C”, “.+G”, “.+I”, “.+T”, “.+Y”, “.+U” to change the atom type to NUC (nucleotide).

If the atom represents an N-terminal attachment (ACE, MYR, etc.), then the atom type is changed to NTER_ATTACHMENT and the residue number is set to one less than the following amino acid.

N-terminal attachments are identified by IsNterModificationo. This routine makes a 2-stage test. First it calls IsNterModType( ) to see if the residue is one of the possible NTer modifications types (currently: ACE, MYR, CBX, FOR). It then looks to see if it is bound to the nitrogen of the following residue.

SetMetals( ) sets the atom type field for metals. Atoms with the PDB file defined type of HETATM are searched and then checked against a list of non-metals in something approaching the order of likelihood of their occurrence. Noble gases are not included since if these are found they will be unbound and can be treated as metals. If the atom is not “C.”, “N.”, “O.”, “S.”, “P.”, “CL”, “BR”, “I.”, “F.”, “B.”, “SI”, “AS”, “SE”, “TE”, “AT”, then the program assumes that it is a metal.

Bad atom names are then corrected: “CAL.” is changed to “CA..” (see, for example, 1ajk), “.MT.” or “MT..” is changed to “HG..” (see, for example, 1bnv).

The justification is then fixed for common metal names: Mg, Zn, Hg, Ca, Mn, K, Fe, Co and Mn.

If the atom appears not to be a metal, then a special check is performed on an element identified as CA (alpha carbon) within a HET group. It is possible that this is a Calcium which was wrongly justified and the element type was assigned by us as C. If the previous and last atoms have different residue numbers, this can be assumed to be a lone atom and therefore to be a Calcium. Similarly, the special case is checked for an Iron atom that is not left-justified and therefore set as F for fluorine in the element type.

SetConnect( ), which checks the CONECT records and adds any missing connectivity, also sets atom types for atoms whose type needs to be changed as a result of that connectivity. (i.e. for BET groups which are bound to protein/nucleic acid and are therefore modifiers of standard residues or are bound het groups).

The distinction between a residue modifier and a bound HET group is made simply on the basis of the residue identifier: if the residue number and chain name of the HET group are the same as the residue to which it is bound then it is a modifier (MODPROT or MODNUC as appropriate); if either is different then it is a bound ligand. Also, if it is a polymer it is always set to be a bound ligand. See “Connectivity” below for details.

SetNSResidues( ) sets atom types for non-standard residues. The BOUNDHET atoms are examined and changed to NONSTDAA/NONSTDNUC if they are linked via the backbone. A connection to the residues N or P, or the preceding residues C or O3* is checked.

1.1.1.2.1.4. Setting Element Types

The element type for each atom is set using SetAtomElement( ). Newer PDB files contain this information already and we take this if it is given. Errors such as F (Fluorine), instead of Fe (iron) are corrected by the SetMetal( ) code.

In brief, the SetAtomElemen( ) routine performs 2 levels of check for valid and unusual atom types and also looks for unusual elements—if the atom name and residue name do not match, then the program checks the second letter and if that is a legal atom name (C,O,N,S,H,P) substitutes that. In the second round of substitution checking where the atom name and residue name do not match, if the last 2 characters of the atom name are digits, then the first letter is checked and if that is a legal atom name (C,O,N,S,H,P), is substituted. Valid and common CA and CD atom names which are followed by two digits are checked. If the atom name is not a subset of the residue name then it is more likely to be a carbon.

In detail, we take the first 2 characters from the atom name that should be the element type. If the first character is a digit then we remove it.

A valid element is defined by the routine ValidElemen( ) that checks against all elements in the periodic table plus “D” (deuterium). If the first two characters do not represent a valid element, then the error is most likely to be an illegal use of the first column of the atom name. So, a warning is inserted and the first column blanked. If after blanking the first column it is still not a valid element, then the entry is replaced with a question mark. This will occur with ASN/GLN where we get “.A[DE][12]”

Otherwise, if it is a valid, but unusual, element it may well be an error (i.e. the atom field being used incorrectly). Such odd elements should have the same atom name and residue name (as they are likely to be unusual metals).

Unusual/odd elements which are possible errors (i.e. common 1-letter elements with another character) are defined by the routine OddElemen( ). This contains a list of all the two letter element names which contain one of the letters C,N,S,O,H, but with more common elements such as cadmium (CD), calcium (CA) and mercury (HG) removed. This gives a warning where atoms have been mislabelled and other similarly wrongly-justified atoms. The element identification routine makes an additional check for common mis-labelled elements followed by 2 digits (such as CD41 [cadmium] instead of 1CD4). The elements listed as “odd” are: “HE”, “NE”, “SI”, “SC”, “NB”, “TC”, “RH”, “SN”, “CE”, “ND”, “HF”, “OS”, “PO”, “RN”, “AC”, “TH”, “NP”, “CM”, “CF”, “ES” and “NO”

If the atom name and residue name do not match then the second character is checked to see whether it is one of the common elements (C,N,O,S,H,P) and if so, this is substituted a warning issued.

If this did not happen, then the 3rd and 4th chars are investigated for digits, in which case the first is a valid element (C,N,O,S,H,P). This check is to ensure that errors such as CE21 (occurring in 8cpa) are corrected.

If it was a valid (and not unusual) element we check for CA and CD followed by two digits. These entries are more likely to be carbons, so we do a check against the residue name and if that does not match, then replace with Carbon.

Checking between atom name and residue name is done by AtomNameMatchesResName( ). Any digits are stripped from the atom name first and spaces are stripped from both. We then see if the atom name is a substring of the residue name.

1.1.1.2.1.5. Connectivity

HETATM connectivity

Having read the connectivity specified in the CONECT records of the PDB file (ParseConec( )), SetConnect( ) verifies and adds to these data. All connection information for BETATOMS and for HETATOM/ATOM connections is stored.

SetConnect( ) also sets atom types except for metals (which must be done previously by SetMetal( ) and the very simple types done previously by SetSimpleAtomType( ).

The CONECT record data is examined to test whether the distances are sensible and to delete any nonsensical entries (>5 Å). Those that refer to models other than the first are deleted, and also those that have NULL pointers as they refer to deleted hydrogens.

Those entries that are between residues (as defined by the residue number and chain name) and involve a metal are also deleted. This is required so as to prevent metals appearing as bound ligands or residue modifiers.

Having deleted connections, any missing ones between HETATM/HETATM, HETATM/ATOM or HETATM/NUC are added. A cut-off of 2 Å is used for bonds only involving organic atoms (N,C,O,S) and 2.5 Å for bonds involving other atoms.

Having obtained a set of connections, the HETATM type is modified to MODNUC/MODPROT (if the residue number matches) or to BOUNDHET (if it does not match). The type is always set to BOUNDHET if the molecule is in a polyHET (checked by IsInPolyHet( ))

IsInPolyHet( ) looks ahead at the next residue to see if this residue is connected to it. This is used to see if an atom is part of a residue in a polyHET. If either the current residue or the next residue has<MIN_ATOMS_IN_RES (3) atoms, then they are not a true polyHET.

The connects are then run through, iteratively changing types that are connected to a MODPROT, MODNUC or BOUNDHET, since all these connected HETATMs should also be of that type.

Finally, ShuffleHetatom( ) is used to move those atoms identified as residue modifications into position in the main list. This is done by looking for the attached residue (which must be of type ATOM or NUC to prevent shuffling within the modified residue) and moving this atom to the end of that residue.

Disulphide information in the PDB file is ignored. Instead, this is done from basic principles: SetDisulphides( ) looks for CYS-SG pairs within 2.25 Å. The ideal disulphide S-S length is 2.03 Å.

1.1.1.2.1.6. Checks for Bad Files

Two routines are used to check for errors in the file: CheckForBadFile( ) and CheckForBadNTerModifier( ).

CheckForBadFile( ) makes the following checks: (1) The same protein/nucleotide residue ID appearing more than once, (2) 3D overlap of 2 chains, (3) HET residues clashing in 3D, (COMPILE-TIME OPTION: (4) HET residue ID appearing more than once).

First entries are checked for a residue identifier that appears twice. This generally indicates multiple models without model records. It can also indicate alternate conformations that have been placed at the end of the file without the alternate indicator column set in the PDB file instead of as part of the residue concerned. Note that HETATMs are not checked, since these could be residue modifications and therefore have the same identifier. At the same time, the box boundaries of each chain are recorded.

The program steps through each residue (amino acid or nucleotide) and then steps through each other residue (amino acid or nucleotide). If the residue and chain names match then there is an error.

Chains that overlap in 3D are then checked for. First, the bounding boxes are checked to see if the CofGs (centres of gravity) are within 10% (1% for nucleotides) of another chain's bounding box smallest dimension. If they may clash on this basis, then a VDW overlap check is performed with the ChainsClash( ) routine. This simply checks for more than MAX_VDW_CHAIN_CLASH (100) clashes of less than VDW_CLASH_SQ½(2.7) Å.

Next het groups (but not water) that clash within a het chain are checked for. ResiduesClash( ) looks for more than MAX_VDW_RES_CLASH (32) clashes of less than VDW_CLASH_SQ½(2.7 Å).

If the compile time option CHECK_DUPE_HETIDS is defined then duplicate HET residue numbers are checked for.

Residues such as ACE and MYR are generally (but not always) N-terminal additions. When they are N-terminal additions, they should be placed at the start of the chain they modify, but sometimes they are erroneously placed in with the HETATMs after the chain. If this is so, then the code will have identified them as BOUNDHET rather than NTER_ATTACHMENT.

CheckForBadNTerModifier( ) looks for molecules in the molecule list which are possibly N-terminal modifiers and thus have been listed as HETATMs which are then found to be bound ligands. This element of the program tests if they are actually bound to a Nitrogen and if so, flags this as an error. The routine walks through the molecules list and checks for whether the molecule is one of the possible NTer modifiers. If it is, then the program investigates whether it is labelled as a bound het group (i.e. the molecule type is set to “boundhet”, in which case it is likely to be an error). Each atom in the molecule is then checked in the connects to see what it is bound to. If it is bound to a nitrogen, then it must be an N-terminal modifier and an error is thrown asking the user to move the residue to the correct position in the PDB file.

1.1.1.2.1.7. Sequence Data

The sequence from the SEQRES records is read by ParseSeqres( ) (see above).

The sequence from the ATOM records is read by SetAtomSequence( ). This reads and stores the sequence from the ATOM records (i.e. the ATOM and NUC atom types). Since atom types have been set by this stage, NONSTDAA, NONSTDNUC and NTER_ATTACHMENT are allowed. The sequence is defined by looking for changes in the residue number or chain label.

The ATOM sequence and SEQRES sequence are aligned with DoSeqAlign( ). This also detects errors where a non-standard amino acid has been included in the SEQRES records but not given an individual residue number in the ATOM records. This commonly occurs for N-terminal modifiers, but this situation is handled automatically and the residue number for the N-terminal modifier is reset.

1.1.1.2.1.8. Molecule List

This identifies all individual molecules within the structure.

An incremental molecule ID is assigned to each protein chain and for the HETATM molecules. This label is applied to each residue. For each chain (judged by the chain label), the program checks whether standard ATOM records (protein or nucleotide) are present. If so, then a molecule entry is created for the chain calling it “protein” or “nucleic”. (SetMoleculeType( )) (see below for details).

If the ATOM record “protein” is present, then it is checked whether it is really a peptide rather than a protein chain (CheckForPeptide( )). A peptide is defined as having<30 residues. Next it is checked whether it is Cα-only (CheckForCAOnly( )) and the label is changed from “protein” or “peptide” to “caprotein” or “capeptide”. Cα-only is defined on the basis of atom count being less than twice the residue count.

The chain is then worked through again, one residue at a time, looking for HETATMs. For each residue in this chain, if the flag has not been set to say that this residue has been used, then a new molecule entry is created. Again, when the new molecule is created, SetMoleculeType( ) is called to fill in information about the molecule type (see below for details). All het residues which are linked to this current residue (via CONNECT information) are then also marked as a member of this new molecule. When a link is found, the routine is called recursively to mark HET residues connected to that one. If any such additional residues were found, then the polymer flag is set for the molecule.

Protein/nucleic chain molecules are given the chain label as a name, while non-protein molecules are given the first residue name. This occurs as part of the CreateMolecule( ) routine. Finally, the molecule list is run through, resetting names for the polymers and peptides (SetPolymerName( )). If the entry is a peptide, the program starts from the first residue and keeps appending residue names until a ligand or a change in chain label is reached. If the entry is a polyHET, all the atoms are run through, starting from the first residue, looking for all those residues assigned to the same molecule and appending their residue names.

In SetMoleculeType( )), the atom type of the first atom in the molecule is checked. If this is one of MODPROT, ATOM, NONSTDAA, NTER_ATTACHMENT, then the molecule type is set to “protein” (a check is performed later to see if is actually a peptide or a Cα-only version of protein or peptide). Default name is the chain name (modified later if it's a peptide).

If it is one of MODNUC, NUC, or NONSTDNUC, then the molecule type is set to “nucleic”. The name is set to the chain name.

If it is METAL, then the next atom is checked in order to see if it is in the same residue and, if so, set the molecule type to “metalcplx”; otherwise it is set to “metal”. The name is set to the residue name.

If it is HETATM, then the type is set to “het” and the default name is set to the residue name (modified later if it's a polyHET).

If it is WATER, then the type is set to “water” and the default name is set to the residue name.

If it is BOUNDHET, then the type is set to “boundhet” and the default name is set to the residue name (the name is modified later if it's a polyHET).

The complete list of valid molecule types is:

protein A protein molecule (>30 residues)

caprotein A Cα-only protein molecule (>30 residues)

peptide A peptide molecule (≦30 residues)

capeptide A Cα-only peptide molecule (≦30 residues)

nucleic A nucleotide molecule

metal A metal ion

metalcplx A metal complex

water A water molecule (This is a compile-time option—normally water molecules are not listed in the molecule list.)

het A het group

polyhet A polymer het group (e.g. a sugar chain)

boundhet A het group bound to the protein. (May be a single residue or a polyhet—the name given will distinguish.)

1.1.1.2.2. solv

Determine residue accessibility of structure in xmas file using the method published by Lee and Richards (1971), Journal of Molecular Biology 55: 379-400. Append information to xmas file.

1.1.1.2.3. ss

Determine secondary structure of protein described in xmas file. Kabsch - Sander algorithm is used (Kabsch W, & Sander C (1983) Biopolymers 22: 2577-2637). Append information to xmas file.

1.1.1.2.4. hb

Predict inter-structure hydrogen interactions of protein described in xmas file, based on protein strucure and bonding geometry, according to Baker and Hubbard (1984) Progress in Biophysics and Molecular Biology, 44: 97-179. Append information to xmas file.

1.1.1.2.5. ligplot

Generate a two-dimensional diagram of protein-ligand interactions; create a file loadable by LigEye to view this diagram. ligplot is an external utility written by Roman Laskowski (Wallace et al., (1995) Prot. Eng. 8: 127-134).

1.1.1.2.6. Load pdb

Load pdb information, secondary structure information, residue accessibility, from xmas file into CARSS database.

The following xmas entries are loaded:

| header | molecules | atoms | experimental | het | atomseq | miscellaneous |
|---|---|---|---|---|---|---|
| pdbcode | type | resnum | type | text | sequence | atomseq |
| name | name | resnam | resolution | resnam | chain | source |
| date | id | chain | rfactor | | | keywords |
| | chain | molid | freerfactor | | | |
| | | resaccess | | | | |
| | | ss | | | | |
| | | type | | | | |

The atomseq is checked against the residues for consistency.

Individual residues in each sequence are sorted into order of occurence and assigned a consecutive sequence order number to reflect their logical position in sequence, in addition to their PDB number and insertion code.

Modified residues are inserted by reference to the main-chain residue to which they are attached.

The source records are stripped of any surrounding double-quotes and MOL-ID section.

The references are parsed before insertion into the database.

The resnum records are parsed into number and insertion code.

1.1.1.3. Load SWISS-PROT (http://www.expasy.ch/sprot/sprot-top.html)

Load SWISS-PROT information into CARSS database.

The following SWISS-PROT entries are loaded:

| Information | Records |
|---|---|
| Entry Name | ID |
| Accession Number | AC |
| Date | DT |
| Gene Name | GN |
| Keywords (structure and function) | GN |
| Organism Species | OS |
| Bibliography | RN, RX, RA, RT, RL |
| Seqeunce | SQ |
| Comment Block | CC |
| Feature Table | FT |

Notes:
Only MEDLINE codes from the Bibliography RX records are used.

1.1.1.4. Load PROSITE (http://expasy.hcuge.ch/sprot/prosite.html)

Load PROSTE; profiles into CARSS database.

The following PROSITE entries are loaded:

| Information | Record |
|---|---|
| Entry Name | ID |
| Entry Accession | AC |
| Creation Date | DT |
| Description | DE |
| Motif Regex | PA |
| Documentation Reference | DO |

1.1.1.5. Load PRINTS (http://iupab.leeds.ac.uk/bmb5dp/prints.html)

Load PRINTS information into CARSS database, linking against information from SWISS-PROT.

The following PRINTS entries are loaded:

| Information | Record |
|---|---|
| Entry Name | gc |
| Entry Accession | gx |
| Creation Date | ga |
| Fingerprint Title | gt |
| Bibliography Information | gr |
| Annotation Text | gd |
| Motif Accession | fc |
| Motif Title | ft |
| Motif Matches | fd |

Notes:
Bibliography information is parsed into serial number, author, title, and publication details.

1.1.1.6. Load Enzymes (http://www.expasy.ch/enzyme/)

Load enzyme information into CARSS database, linking against information from SWISS-PROT.

The following enzyme entries are loaded:

| Information | Record |
|---|---|
| EC Number | ID |
| Enzyme Name | DE |
| Alternate Enzyme Name | AN |
| Catalytic Activity | CA |
| Cofactors | CF |
| Comments | CC |
| Associated Disease | DI |
| PROSITE X-Refs | PR |
| SWISS-PROT X-Refs | DR |

1.1.1.7. Update SWISS-PROT Taxonomy

Link SWISS-PROT entries against taxonomy entries in CARSS database.

1.1.1.8. Update pdb Taxonomy

Link PDB entries against taxonomy entries in CARSS database. Uses taxonomy assignments from Steve Bryant at NCBI to map tax IDs to PDB chains (ftp://www.ncbi.nlm.nih.gov/mmdb/pdbeast/table).

1.1.1.9. Load pdb Interactions

Load PDB interaction information (as generated by hb), residue accessibility (as generated by solv) and secondary structure information (as generated by ss) into database, linking against PDB sequences.

1.1.1.10. Load genbank

Load genbank information into CARSS database, linking against enzyme and taxonomy entries.

The following Genbank entries are loaded:

| main | CDS | source |
|---|---|---|
| LOCUS (Locus) | db_xref | organism |
| LOCUS (Date) | EC_Number | cell_line |
| ACCESSION | function** | cell_type |
| DEFINITION | gene* | chloroplast |
| KEYWORDS | map* | chromoplast |
| NID | note** | chromosome |
| VERSION (Accn Versn) | product | mitochondrian |
| VERSION (NID) | standard_name | clone |
| REFERENCE | protein_id | db_xref |
| AUTHORS | translation | germ-line |
| TITLE | | haplotype |
| JOURNAL | | map* |
| MEDLINE | | note* |
| | | plasmid |
| | | proviral |
| | | virion |
| | | tissue_lib |
| | | tissue_type |

Notes:
The NID will have the preceding code letter removed so that it is just a number.
*Multiple record strings are concatenated together.
At least one of VERSION (Accn Versn) or NID must be present.
VERSION (NID) is used only if NID is not present.
Both incarnations of db_xref are parsed into database code & accession.
Multiple entries marked with* are concatenated.
product and standard_name records are treated together; the first two are loaded, subsequent occurences of either are ignored.
Only one of choloroplast, chromoplast, chromosone and mitochondrian are used.
Any source:map entries are concatenated onto any CDS:map entries
Only one of proviral or virion should be present.

1.1.2. Process Sequences

Cross-reference PROSITE database with primary database (Genbank, PDB, SWISS-PROT). Compare sequences, grouping similar sequences for later comparison.

1.1.2.1. Export Extracted Sequences

Export collated sequence database into FASTA files. These sequences are all those imported from Genbank, SWISS-PROT or PDB.

1.1.2.2. PROSITE Profile Matching

Generate matches of collated sequences against PROSITE regular expressions and profiles.

1.1.2.3. Generate nr Sequences (Dunce)

Group sequences from the primary databases according to close similarity to reduce the load of later comparisons. This is necessary because these databases include a large amount of redundant sequence information that would clog up the database and considerably slow analysis of the data contained within it. Redundancy is performed using a novel algorithm that is implemented using a program referred to herein as DUNCE.

The Dunce program reads one or more files containing genetic sequence data in FASTA format and rewrites the data as a non-redundant data set in FASTA format to the standard output. Only input sequences that are not contained within other input sequences will be copied to a new FASTA format file. Subsequences that are not output have their positions relative to output sequences stored. In addition, if multiple identical sequences occur in the input data, only the first one encountered will be a candidate for the output data set.

Dunce has been written to run quickly with large numbers of input sequences, at the expense of requiring large amounts of memory. Using this program, over 400,000 sequences have been processed in 15 minutes on a Sun Ultra Sparc with 1 Gbyte of memory.

The Dunce program finds matches by splitting sequences into contiguous, non-overlapping fragments that are placed in a hash table. Then every possible (overlapping) fragment from each sequence is matched against the hash table to find possible matches. Candidate matches for a given sequence are found by comparing fragments against the hash table. If two fragments from different sequences match in the hash table, the complete sequences are checked against each other character by character.

1.1.2.3.1. Details of the Algorithm:

Let each sequence S consist of letters S[i], with i from 1 to L_{S,} where L_{S} is the length of sequence S.

Each sequence is processed sequentially. As each sequence is processed it is split into overlapping fragments of a particular word size, K, which is a configurable at run time. The default setting for word size K is 10. The fragments will consist of characters as follows:

| Fragment number | Characters from sequence |
|---|---|
| 1 | S[1] . . . S[K] |
| 2 | S[2] . . . S[K + 1] |
| . . . | . . . |
| L_{S} − (K + 1) | S[L_{S} − (K + 1)] . . . S[L_{S}] |

By default, any sequence less than 30 characters long (default value, 30 residues) is rejected and processing continues with the next sequence in the input data set. Again, the length at which sequences are rejected is configurable at run time.

A hash code is calculated for each of these fragments (for reference relating to hashing, see Knuth, The art of computer programming, vol. 3, Sorting and searching, pp 506-549 (Addison-Wesley 1973). For each such hash code, every other sequence containing a fragment of that hash code is considered as a candidate match. For each candidate match, the first thing that is checked is that the point within the sequences at which the matching fragment occurs is concordant with a possible match. The following diagram denotes the matching fragment by the string ABCD:

Sequence A: XXXXXABCDX
Sequence B: XABCDXXXXXXXX

In this example, there is no need for a character by character comparison. Neither sequence can be a subsequence of the other based on the matched fragment.

If the candidate matches are viable, a simple character by character comparison is done. The first and last characters are ignored. This feature avoids potential problems that are caused by, for instance, spurious methionine residues being present at the start of some cloned sequences. It should be pointed out that the ignored residues are NOT deleted from the output data set.

There is also the facility to specify that the Dunce program ignores a given number of internal differences and thus performs only approximate comparisons. A command line flag specifies a so-called “fuzz factor”, given a positive integer parameter that equals the number of individual residue differences within a sequence comparison which will be accepted before the comparison sequences are deemed to differ.

If the sequence being processed is found to be either identical to, or a subsequence of, one already in the hash table, then this fact is recorded, and no further processing is done to this sequence. Processing continues with the next sequence in the input data set.

Alternately, if any of the candidate matches is found to be a subsequence of this sequence, then that is recorded and for each subsequence found, all the corresponding fragments in the hash table are deleted. For any two sequences, one is considered to be a subsequence of the other if it is a strict subsequence (ignoring end residues) with (in the current implementation) up to 3 residue differences. Sequences are therefore grouped together such that within any group, every sequence other than the longest is a subsequence of the longest sequence.

Finally, if no identical or super sequences were found, this sequence is added to the hash table. In distinction to the checking stage above which used overlapping fragments, only contiguous, non overlapping fragments are actually added to the hash table, i.e.

| Fragment number | Characters from sequence |
|---|---|
| 1 | S[1] . . . S[K] |
| 2 | S[K + 1] . . . S[2K] |
| . . . | . . . |
| (n − 1)K + 1 | S[(n − 1)K + 1] . . . S[nK] |

where n=floor (L_{S}/K). The characters in the sequence from S[nK+1] to S[L_{S}] are ignored.

The process is repeated sequentially for all the sequences in the input file(s). There is not necessarily a unique way to reduce a data set through the redundancy process. This is because a sequence can be a subsequence of two other sequences, both of which are mutually distinct. See diagram:

Sequence A: XXXXXABCDX
Sequence B: XABCDXXXXXXXX
Sequence C: ABCD

Sequence C is a subsequence of both A and B, but A and B are mutually unrelated.

A report is produced, specifying for each sequence in turn:

i) Any sequences that this sequence subsumes (if it is the longest in its group), or

ii) The sequence that subsumes this sequence.

In each case, the alignment of the shorter sequence in relation to the longer sequence is specified by index of the start and end of the sequence. This index does include the trailing residues. Indexing is 1-based for this purpose.

The Dunce program copies the header line from the input FASTA file to the output FASTA file almost verbatim, except that it will put a space between what it considers to be the sequence identifier and the rest of the header text. Dunce considers that any text following the ″ character up to the first space or the second ‘I’ character is the sequence identifier.

The Dunce program can accept multiple input files. If a new sequence file or files become available, then it is possible to speed up the process of adding these to a file that is already non-redundant by means of the “update” flag given to dunce at the run time. If this flag is given then the Dunce program will simply add the contiguous fragments of the non-redundant sequences to the hash table, without checking for any matches. If used correctly on a non-redundant file, then of course there would not have been any matches anyway. Only when processing reaches the second and subsequent files will Dunce start checking for hash table matches.

The update flag is only of use to speed up processing, and then only when one file is already known to be internally non-redundant. When correctly used, it has no effect on the actual data that is output.

Dunce Report File Format:

LINE+

LINE: SEQUENCE_NUMBER “\t” ACCN_CODE “\t” LENGTH_SPEC″ (CEDES|CEDED)

CEDES: ‘supersedes’ CEDE_SPEC+

CEDE_SPEC: “†n†t” SEQUENCE_NUMBER “\t” ACCN_CODE “\t”LENGTH_SPEC ‘[’ START ‘:’ END “]\n”

CEDED: 'superceded by’ SEQUENCE_NUMBER “\t” ACCN_CODE “\t” LENGTH_SPEC ‘[’ START ‘:’ END “]\n”

SEQUENCE_NUMBER: \d+

ACCN_CODE: \w+

LENGTH_SPEC: “len=”\d+

START END: \d+

1.1.2.4. Load nr Sequences

Load NR sequences back into CARSS database.

1.1.2.5. PROSITE re Matching

Generate matches of collated sequences against PROSITE regular expressions and profiles.

1.1.2.6. Generate Selected Sequences

Combine the nr sequences and all pdb sequences. These sequences are used by the graphical front end application to provide visualisation structures for arbitrary sequences at a user's prompt.

1.2. Calculate Relationships

Seek relationships between sequences.

1.2.1. Masking

Within each sequence, residues that are identified as being in areas that would likely disturb comparisons are marked for exclusion from subsequent calculations.

1.2.1.1. Export pdb FASTA

Extract all PDB sequences from the database.

1.2.1.2. Export non-pdb FASTA

Extract all non-PDB sequences from the database.

1.2.1.3. Dunce

Elect representatives of all inputs by removing all but one of each group of similar sequences. See section 1.1.2.3.

1.2.1.4. Import pdb nr Sequences

Load sequences elected as representatives of all PDB sequences.

1.2.1.5. Import non-pdb nr Sequences

Load sequences elected as representatives of all Non-PDB sequences.

1.2.1.6. Mask Sequences

Produce a masked version of each sequence of interest.

1.2.1.6.1. Signal Peptide Masking

Mask off signal peptide residues.

For each sequence:

A 20-residue window is scanned along the sequence. Parameters from the MEMSAT algorithm [http://globin.bio.warwick.ac.uk/˜jones/memsat.html; Jones, D. T., Taylor, W. R. and Thornton, J. M. (1994)Biochemistry. 33:3038-3049] are applied to this window to provide a memsat score.

A 30-residue window is scanned along the sequence, unto which is applied a variant of the algorithm desribed by Nielson et al., (http://www.cbs.dtu.dk/services/SignalP/index.html; Nielsen et al., (1997) Protein Engineering 10, 1-6), with the ‘centre’ residue biased to appear at position +25 within this window. This score is converted into a log-probability score.

Each scan is continued up to residue 120.

For each residue, a sum score is found by summing the memsat and log-probability scores (each multiplied by a constant factor).

Any residues for which the sum score is below a predefined cutoff are discarded.

If any residues remain, the residue with the highest log-probability score is selected as the head of the signal peptide.

All residues up to and included the identified signal peptide are masked off.

The cutoff point and score product factors are predetermined by scoring a number of known sequences with identified signal peptide regions taken from SWISS-PROT (version 36), and compared with a number of sequences from the same SWISS-PROT database with no identified signal peptide regions, and which are found only in the cytoplasm or nucleus.

1.2.1.6.2. Low Complexity Masking

Mask off areas of high concentrations of specific residues, where functionality is questionable, and that would bias sequence comparisons.

This consists of three different masking techniques, performed in one step:

1 Local Low-Complexity Masking

In this technique:

A 10-residue sliding window is scanned along the entire sequence.

Over the ten positions, the number of different types of residues are counted (non-standard amino acids are counted as one type).

The window length is divided by the count of distinct residue types, to give an average count of each residue type. If this average is 3 or above, the whole window is masked.

2 Windowed and Sequence Low-Complexity Masking

The distribution of each residue type (non-standard types being counted as a single type) over 100-residue windows and whole-sequences is pre-calculated over a set of approx. 270,000 non-redundant sequences. For each residue type, the mean (μ) and standard deviation (σ) of the distribution is found; and a cutoff value, being μ+xσ is found (where x is 4 for sequence, 5 for window).

A 100-residue sliding window is scanned along the entire sequence.

At each position, the count of each residue type within the window is found. For each residue, if the count of that residue type exceeds the cutoff value for that residue (for windows), then the instances of that residue are masked.

Also, the count of each residue type is found over the whole sequence. For each residue whose count exceeds the sequence cutoff for that residue type, residues of that type are masked over the whole sequence.

1.2.1.6.3. Coiled-Coil Masking

Mask off coiled-coil areas that are of questionable functionality.

Identification of coiled-coil areas is done using the algorithm developed by Lupas et al, 1991 (Predicting Coiled Coils from Protein Sequences, Science 252:1162-1164; http://www.isrec.isb-sib.ch/software/COILS_form.html), using the MTIDK matrix over a 21 residue window, without the extra weighting for coil positions ‘a’ and ‘d’, to give a per-region probability score.

If a region has a probability score of greater then

it is masked.

1.2.1.6.4. Membrane Masking

Mask off protein areas that are identified as being within cell membranes.

Identification of membrane areas is done using the algorithm described by Jones et.al. (1994) Biechem. 33: 3038-3049. This algorithm gives a list of potential candidates for transmembrane regions together with a score for each region as well as on overall score.

If the overall score is greater than 8.0, or if the overall score is greater than 3.0 and an individual region gets a score greater then 0.5, then the sequence is masked appropriately.

1.2.1.6.5. Combine Masks

Combine masks by masking off each residue that is identified in one or more of the individual masking steps.

1.2.1.7. Load Masking

Load information into database describing results of all sequence masking performed.

1.2.2. Inpharmatica Genome Threader

Look for similarities between sequences of known and unknown structure by taking into account both sequence and structure information.

1.2.2.1. Select Training Sequences

Select sequences to use for training Inpharmatica genome threader to distinguish true and false relationships. True and false relationship lists are generated by taking data from a classification of known structures where distant relationships have been identified on the basis of similar structure and function and are clustered together. We use the C.A.T.H. structure classification of Orengo et al., 1997 (Structure 15;5(8):1093-108) by selecting the C.A.T.H numbers from the appropriate sequences. This classification scheme has many levels of hierarchy. However, for the purpose of this work, only five are important to us: Class, Architecture, Topology, Homologous superfamily and Sequence.

Each domain of a protein is ascribed a set of numbers. Using the above description, if two protein domains have identical classification numbers at all five levels, they are perceived to have a greater similarity than if only four or less match. The classification is hierarchical in that while 1.2.3.4 and 1.2.3.1 match at the CAT level, 1.2.3.4 and 2.2.3.4 have no similarity.

If the C.A.T.H numbers match then it is considered to be a true relationship, if the C.A.T. numbers differ then it is a false relationship. However if the C.A.T numbers match but the C.A.T.H numbers differ then these are considered to be indeterminate and not used for training the network. In detail, the lists are generated by the following steps.

1. Discard all CATH 4.X.X.X entries

2. Discard all CATH entries of obsolete PDB files

3. Select only CATH entries which consist of a single domain >=40 residues or domains from a multi-domain protein where there is only one contiguous region >=40 residues

4. From this reduced list, select the longest representative for each S family

5. Split into two groups:

Topologies with only one H level

Topologies with multiple H levels

6. Make pairs of known matches as follows:

A pair of sequences are related if they have the same CATH number

A pair of sequences does not match if they have different CAT numbers

7. A pair of sequences are indeterminate if they have the same CAT Number but different CATH numbers

8. Discard the indeterminate pairs

9. Reduce the numbers of pairs of known non-matches to a reasonable number:

For single H topologies, use only the pairs generated by the 4 longest representatives from each A level.

For multiple H toplologies, use only the pairs generated by the 3 longest representatives from each T level.

1.2.2.2. Train gt Net

Train the neural network using selected training sequences. The network is trained against the selected sequences using the neural-networking standard back-propagation method.

The neural network used consists of a 3-input I-output single hidden layer system using the standard back-propagation method for training (see Rumelhart, D. E. and McClelland, J. L. (1986): Parallel Distributed Processing: Explorations in the Microstructure of Cognition (volume 1, pp 318-362). The MIT Press). The training set is therefore based on a selection of relationships from all of the possible combinations from the set of selected structures.

Confidence Prediction

Once the network has been trained and a score obtained for all of the relationships it is possible to put the scores into bins and count the number of true and false relationships that achieve a given network score.

This then allows for the network score to be converted into a confidence value that gives the percentage probability of a relationship being correct if it achieves a given network score.

1.2.2.3. Create pdb esn equivs

Generate mapping from PDB accession codes to database-internal unique sequence IDs.

1.2.2.4. Split FASTA

Split masked sequence database into smaller chunks.

1.2.2.5. Partition Profiles

Split PSI-BLAST profiles database into smaller chunks.

1.2.2.6. Do gt fwd/rev

### SUMMARY

Control mechanisms of sequence comparison, alignment and structure overlay.

The method starts by taking a representative set of known three-dimensional structures and calculating statistical potentials for the residues and interactions.

The first potential considers the accessibility or solvation potential for a given residue type. This is the area of a residue's side chain that is accessible to a solvent such as water.

The second is the distance between atoms within pairs of residues, also taking into account the linear separation of the residues along the protein chain and the local secondary structure of the residue.

This set of statistical potentials need only be calculated once, with the subsequent calculations making use of these pre-calculated values.

The sequence of unknown structure (the query sequence) is then in turn aligned against sequences from proteins of known structure. As the skilled reader will appreciate, this can be done using any alignment procedure. In a preferred embodiment, both a local and local-global dynamic programming algorithm are used.

Once the query sequence has been aligned against a known structure, the recalculated potentials for finding the residues from the query sequence in that conformation are summed along its protein chain. This provides total energies for both the salvation and pairwise interaction.

These two potentials, along with the score from the alignment stage are then passed through a neural network to give a single score value. The neural network is trained on a set of known structures.

In order to aid interpretation, the results from a set of known structures that have been analysed according to the above procedure are assessed to produce a mapping from the neural-network score to a confidence value. This expresses the results from the algorithm as the probability of the unknown sequence having the same structure as that of the structure to which it was compared.

Selection of Representatives

In order to calculate the statistical potentials, an unbiased selection of the available structures must be used. This was done as described above using the CATH database. The selection was done by choosing the longest representative chain for each C.A.T.H.S value.

Calculation of Solvation Potentials

The calculation of the accessibility or solvation potential first requires each residue in the chosen structures to have its accessibility calculated. This may be done using an implementation of the Lee and Richards algorithm (Lee and Richards (1971) JMB 55: 379-400), though any other suitable algorithm may be used that achieves the same result.

The residues are then collected into accessibility groups, with each group spanning 10% of the range (i.e., 0-10% accessibility, 11-20% accessibility, etc.). The total number of occurrences for each residue type is then counted over each bin.

The statistical potential for a residue's accessibility may be calculated as in Equation 1 below:

$${E}_{r} ( a ) = - RT ln \frac{{f}_{r} ( a )}{f ( a )} \qquad ( 1 )$$

where r is the residue type, a is the accessibility bin and Eᵣ(a) is the potential for a given residue to have a given accessibility.

The values fᵣ(a) and f(a) are the relative frequency of residue occurring with accessibility (a), and the frequency of any residue occurring with accessibility (a), and are calculated as follows:

fᵣ(a)=Nᵣ(a)/Nᵣ (2)

f(a)=N(a)/N (3)

where Nᵣ(a) is the number of residues of type r with accessibility (a), Nᵣ is the number of residues of type r, N(a) is the number of residues with accessibility (a) and N is the total number of residues.

Calculation of Pairwise Potentials

The calculation of pairwise potentials is similar to the calculation of the solvation potentials, but with some differences. Firstly there are five potentials to be calculated, one for each of the five atom pairs (Cβ-Cβ, Cβ-N, Cβ-O, N-Cβ, O-Cβ) between the two residues.

Secondly, there is an additional parameter or state for the atoms; this is the linear separation along the protein chain. Finally, the distance between the atoms is used in place of the accessibility.

As before, the values are placed into bins, which are calculated as follows. The distances between atoms are placed into 1 Å bins, with any distance greater than 40 Å placed into a single bin. The linear separation of residues are placed into bins as follows. If the separation is 10 or less then each value is placed into its own bin. Linear separations between 10 and 30 are placed into another bin, and any separations over 30 are placed into another separate bin.

As before, all residues within the chosen structures are considered, but as these are interactions between pairs of residues they are only calculated between all possible pairs of residues along unbroken lengths of protein chain.

The potentials are calculated as follows

$${E}_{{rr}^{′}}^{S} ( d ) = RT ln ( 1 + {N}_{{rr}^{′}}^{s} σ ) - RT ln [ 1 + {N}_{{rr}^{′}}^{s} σ \frac{{f}_{{rr}^{′}}^{s} ( d )}{{f}^{s} ( d )} ] \qquad ( 4 )$$

where s is the linear separation bin and d is the bin for the atomic distances, r is the type of the first residue of the pair and r′ is the type of the second. As mentioned above, five of these potentials are calculated, one for each of the pairs of atoms.

The relative frequencies are calculated as

$$\begin{matrix}\begin{matrix}{f}_{{rr}^{′}}^{s} ( d ) = \frac{{N}_{{rr}^{′}}^{s} ( d )}{{N}_{{rr}^{′}}^{s}} & \end{matrix} & ( 5 ) \\ {f}^{s} ( d ) = \frac{{N}^{s} ( d )}{{N}^{s}} & ( 6 )\end{matrix}$$

Where N^{S}_{rr′}(d) is the number of residue pairs with a given separation and atomic distance, N^{S}_{rr′} is the number of residue pairs with a given separation regardless of distance. N^{S}(d) is the total number of residue pairs with a given separation and atomic distance and N^{S} is the total number of residues with a given separation.

σ

Due to there being so many possible states in which each residue pair may occur, it is possible that a particular state may have very few members, and therefore if the potential was calculated using an equation of the same form as Equation 1, it might not be truly representative. Therefore, the equation has been modified to limit the effect that small numbers of samples can have, with the σ term in Equation 4 controlling this dampening effect.

A number of input file provide sequences for alignment in the Inpharmatica genome threader program, namely “PDB-ESN equivalence list” and “FASTA files”, generated from “Masked sequences” (see section 1.1.2.6); “Profile partitions” generated by partition profiling (see section 1.2.2.5) that is itself derived from analysis of “PSI-BLAST profiles” (see section 1.2.4.1.2) and “FASTA files” (see sections 1.2.1.1 and 1.2.1.2); and the XMAS files generated previously (see section 1.1.1.2.1).

1.2.2.6.1. Align

Look for alignments of maximal similarity between two sequences. Alignment is performed by comparing two sequences, applying a “profile” (mutation potential matrix) to one, and thus looking for areas of similarity between the two sequences. In forward mode, the profile for the structured sequence is used to look for alignments with the other sequence. In reverse mode, the profile for the unstructured sequence is used to look for alignments with the structured sequence. The align programme generates a proposed alignment, and a value representing the confidence of this alignment.

The first alignment is a local alignment using the standard Smith-Waterman dynamic programming algorithm (Smith and Waterman (1981) J Mol Biol, 147:195-197). The second is a local-global method that is similar to the Myers-Miller global alignment (Myers and Miller, (1988) Comput Appl Biosci 4(1):11).

1.2.2.6.2. Structure Overlay

Assess likelihood of protein with unknown structure adopting the structure observed in the aligned portion of the known structure. Each alignment (local, global with forward and reverse) is used, and two scores generated for each alignment: a pairwise energy value, and a solvation accessibility value. The scores are found by summation of potentials over individual residues. The calculation of the potentials is detailed above in section 1.2.2.6.

1.2.2.7. Do gt Net

Each alignment (local and global) is used, and two scores generated for each alignment: a pairwise energy value, and a solvation accessibility value. The scores are found by summation of potentials over individual residues.

To do this, once an alignment has been calculated, the residues of the known structure are replaced by the corresponding residues from the alignment in the sequence of unknown structure. The accessibility potential for each residue is then summed to give a total accessibility score.

Similarly, all of the pairwise contributions from each residue-residue interaction for each of the 5 atom pairs is summed to give a total pairwise energy value. These two values are then passed on to a neural network along with the alignment score.

Three inputs are taken:

Alignment confidence

Pairwise Energy Value

Solvant Accessibility Value

These inputs are supplied to a single-hidden-layer three-node neural network that combines them to a single value, which is compared to the value calculated for a number of known sequence pairings to provide a match confidence value.

1.2.2.8. Combine Local and Global Results (cat)

Combine local and global results.

1.2.2.9. Load Inpharmatica Genome Threader

Load Inpharmatica genome threader results into CARSS database.

1.2.3. Combine Masked Sequences (cat)

Generate a single unified collection of masked sequences from both PDB- and non-PDB sources.

1.2.4. Blasting

Each input sequence is considered in turn, comparing against all other input sequences and looking for areas of commonality.

1.2.4.1. blastseq

1.2.4.1.1. blastpgp

Search for matches to a given sequence.

Given a query sequence, and a database of target sequences, search for sequences with similar portions to the query sequence. Generate a sequence profile if a sufficient number of hits are found for this profile to be meaningful. The profile is a matrix describing the probability of mutation of individual residues in a sequence based upon the presence of alternate residues in similar contexts in other sequences that were identified by the database search. This profile is then used to research the database to identify additional related sequences. If more are found, then a new profile is generated for a further round of searching. This procedure can in principle be continued until convergence, though in practice, an upper limit is set due to restrictions in cpu time available.

Algorithm Used: PSI-BLAST [(Position-Specific Iterated BLAST): Altschul et al., 1997, Nucleic Acids Res. 25:3389-3402]; being a variant on BLAST [(Basic Local Alignment Search Tool): Altschul et al., (1990) J. Mol. Biol. 215:403-10].

1.2.4.1.2. Select Profile

Select a PSI-BLAST “profile” for each given sequence.

Select the profile generated by the last iteration of blastpgp if available, else use the blosum matrix to generate a default profile.

1.2.4.2. psiparse

Extract and reformat hits details from a blastpgp results file. The results are output in the following format:

The first line states the total number of iterations that blastpgp performed.

Subsequent lines each specify a hit, as space-separated columns:

1. The name of the sequence hit.

2. The length of the match.

3. The hit “bit score”: a score of the profile generated by this hit.

4. The hit “e-value”: a normalization of the “bit score”, representing the confidence of the hit.

5. The number of identical residues found in the match.

6. The number of positive scoring (likely mutation) residues found in the match.

7. The index of the starting residue of the match in the subject sequence.

8. The index of the ending residue of the match in the subject sequence.

9. The index of the starting residue of the match in the sequence found.

10. The index of the ending residue of the match in the sequence found.

11. The DNA match frame. Currently unused.

12. The PSI-BLAST iteration in which the match was found.

1.2.4.3. docluster

Sequence matches are clustered using a clustering program that identifies related sequences produced in the blasting step and assigns them to a particular family. The algorithm used describes a method for combining multiple results from one or more sequence database searches into a single result for each distinct ‘hit’. For example, when performing a database search using an iterative algorithm such as PSI-BLAST, the alignment and E-Value may change between iterations, but it still ‘describes’ the same basic region of similarity between the two sequences.

This algorithm described below provides an automated method for finding and producing these similar regions from sets of individual sequence alignments.

Sequence Alignment Values

When two sequences are aligned, regardless of the algorithm used, the resultant values can be split into two groups.

The first group contains those values describing the location of the aligned region of the in two sequences which shall be called sequence A & sequence B. These alignment results can always be represented by four numbers, as gaps in the alignment are not taken into consideration.

The first two describe the extent of the aligned region on sequence A, denoted as [F_{A}, T_{A}] (where F represents “from” and T represents “to”) , and the second two are the extent of the aligned region on sequence B, denoted by [F_{B}, T_{B}]

The second group contains those values which are related to the score or scores produced by the alignment algorithm. For example this algorithm was developed to be used with the output from the PSI-BLAST algorithm (Nucleic Acids Res Sep. 1, 1997;25(17):3389-402), and the values that were used from its output were the E-Value and the iteration number.

Combining Regions

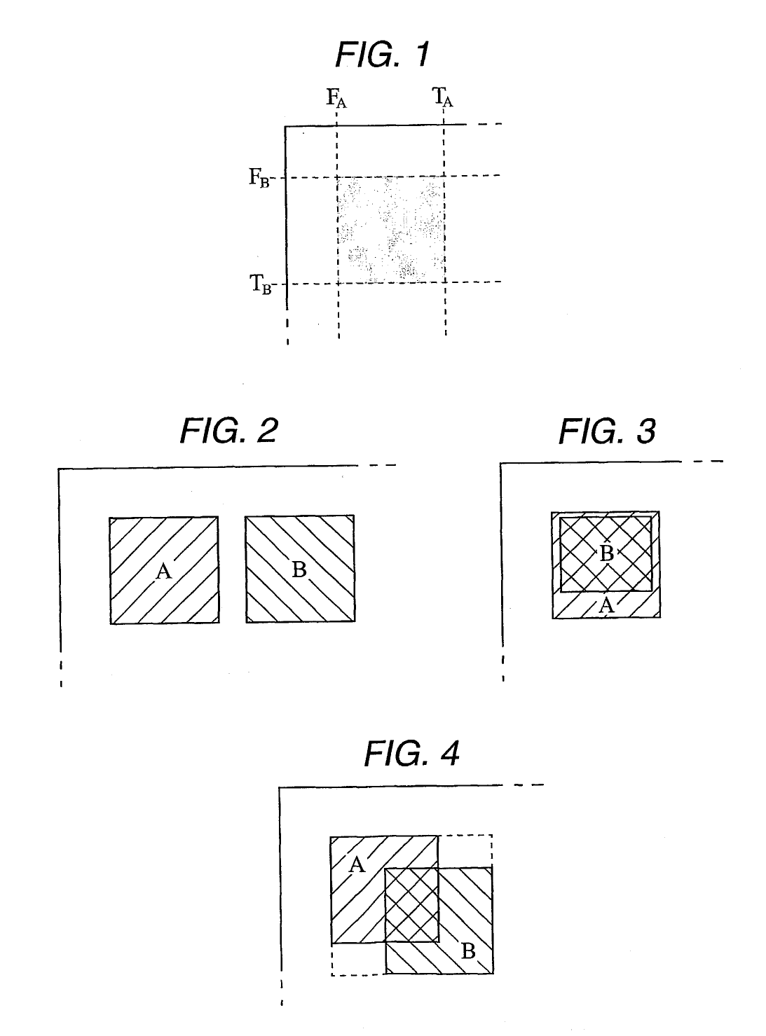

To describe how it is decided if two alignments can be combined into one, the representation shown in FIG. 1 will be used.

The horizontal axis represents the residue numbers from sequence A, and the vertical axis from sequence B. It can be seen that if perpendicular lines are drawn from the position of four numbers representing the alignment, then that alignment region is represented by a rectangle.

In considering two alignments, and whether or not they can be combined into one, there are three possible cases.

In the first case (FIG. 2), the two regions are disjoint, and so the two alignments can be trivially rejected as candidates for being combined.

In the second case (FIG. 3), one region is completely enclosed within another. These two alignments are therefore suitable for merging, with the new representative being the larger of the two regions.

Finally there is the case where the two regions intersect (FIG. 4). The decision on whether these two regions should be merged is based on the area of the intersection. If this area is greater than or equal to 90% of the area of the smaller of the two regions, then the regions are merged.

The value of 90% can of course be varied to suit the particular requirements of the analysis being carried out, but this figure was chosen as it worked well for the combination of results from PSI-BLAST.

If the two regions are suitable for merging then the combined region then becomes the bounding box of the two rectangles. (Represented by the dashed line in the figure.)

Multiple Alignment Regions

In the case where there are multiple alignment regions, e.g. we have one from each iteration of the PSI-BLAST algorithm, the above calculations must be repeatedly performed many times, continually merging alignments together until no more candidates for merging are found. Finally there will then be one alignment representative for each distinct region of the sequences that can be found. In order to perform this procedure efficiently two things should be done. Firstly, one of the standard ‘subset construction’ algorithms should be used, this will minimise the number of comparisons that need to be done between alignment pairs.

Secondly, it should be noted that in the previous section the example in which one region is completely enclosed by another is shown as a completely separate case. However in reality it is just a special case of two regions intersecting, in which the area of overlap must be greater than 90% of the smaller rectangle.

The reason for showing it as a separate case is that it is much easier to calculate than the general overlap case. Therefore, if all of the enclosed alignments are removed first, there are less alignments to compare afterwards, speeding up the calculation.

Alignment Values

In the above sections on merging regions, no mention was made of what to do with the alignment values. This is because it is independent of the merging procedure and can be changed to suit the particular application.

In the case of merging results from PSI-BLAST, the values that were of particular interest were the iteration number and the E-Value combination. These were required for the first and last iterations in which an alignment occurred, as well as the best E-Value that was achieved.

When two regions were being merged using the above criteria, the lowest and highest iteration/E-Value pair present in the two alignments was stored in the combined alignment, along with the lowest E-Value achieved by either of the two alignments together with the iteration number this was achieved on.

In use it has been found that the application of this algorithm to the results of a PSI- BLAST search which ran for 20 iterations can reduce the total number of hits to as little as one fiftieth of the original number.

The results are output in the following format:

The first line states the total number of iterations that blastpgp performed.

Subsequent lines each specify a (merged group of) hit(s), as space-separated columns:

1. The name of the sequence hit.

2. The local hit number (such that this, grouped with the name of the sequence hit, are unique for a subject sequence).

3. The length of the match. This is the length of the longest match in the cluster.

4. The bit score of the hit with the “best” e-value.

5. The hit “e-value”: a normalization of the “bit score”, representing the confidence of the hit. This is the best (lowest) e-value over all the hits grouped.

6. The identical residues count of the hit with the “best” e-value.

7. The positive scores count of the hit with the “best” e-value.

8. The lowest index of the starting residue of the matches in the cluster in the subject sequence.

9. The highest index of the ending residue of the matches in the cluster in the subject sequence.

10.The lowest index of the starting residue of the matches in the cluster in the subject sequence.

11.The highest index of the ending residue of the matches in the cluster in the subject sequence.

12.The DNA match frame. Currently unused.

13.The lowest PSI-BLAST iteration of the hits in the cluster.

14.The evalue of the hit of the lowest PSI-BLAST iteration in the cluster.

15.The highest PSI-BLAST iteration of the hits in the cluster.

1.2.4.4. Load PSI-BLAST

Load summarized blast hits into the CARSS database.

1.2.5. Clustering

Identify clusters of similar results from the Blasting step (across different sequences).

The challenge for generating a full multiple alignment that contains all residues from every sequence requires that additional processing must be performed on the previously generated 20×X profile (where X is the length of the sequence). This is because no information concerning the positioning of gaps (required when aligning longer sequences) is contained within the profile. To achieve this the following methodology is followed.

After each pairwise alignment of a sequence to a profile, the profile for the nominated sequence is then modified by the algorithm before being used to produce the alignment for the next sequences. The method of modification is shown in the following section. Areas in the profile which have been modified are marked as such, as they affect the way that an alignment is scored in the dynamic programming step. This procedure is repeated for each sequence in turn until the complete alignment is produced.

If a sequence has previously been aligned by some other method, and it has been discovered that it can align against the nominated sequence in multiple locations, it is necessary to put it through this algorithm multiple times, one for each of these ‘local hits’. The alignment produced for each appearance of the sequence must be constrained so that the correct local hit is chosen, rather than aligning the best area repeatedly. This constraint mechanism can also be used to make sure that particular areas of interest which have been previously identified are preserved by the alignment procedure.

Profile Modification

Initially the profile for the nominated sequence can either come from an iterative algorithm such as PSI-BLAST, or it can be generated for the sequences using a standard scoring matrix such as Blosum-62.

This profile is then used to generate the alignment with a sequence, however after each pairwise alignment is calculated the profile is modified as show in FIG. 1.

Where the alignment calls for a gap in the profile, the profile is modified by inserting the residues from the aligned sequence which match up with the gap. These inserted residues are marked as such, as they have an effect on future alignments as described in the next section. The scoring values which these inserted residues are given are taken from a standard matrix such as Blosum-62.

Alignment Procedure

As described above, the alignment procedure is based on a standard dynamic programming algorithm. However the following changes have been made.

When residues in the alignment have a negative score, and lie within one of the inserted regions of the profile their score is reset to zero. This has the effect of allowing multiple sequences which have similar regions, but which were not in the original profile, to all be aligned together without penalty, while at the same time still increasing the score for correctly aligned regions which have a positive score.

Also, whenever the alignment procedure requires a gap to be inserted or extended into the sequence which is being aligned, then if the area in which it is being placed corresponds to one of the inserted regions in the profile, it is done without penalty. This has the effect of allowing a sequence which would normally align against the profile without the need for a gap, to be aligned without an inserted region interfering.

Constraining Alignment

As described above, there are a number of reasons to constrain the alignment of each sequence to a particular region. This can be done by excluding regions from consideration by the dynamic programming algorithm by setting the scores in the excluded region to be extremely negative, beyond what could occur naturally during the execution of the algorithm, normally the largest negative value which can be stored by the computer.

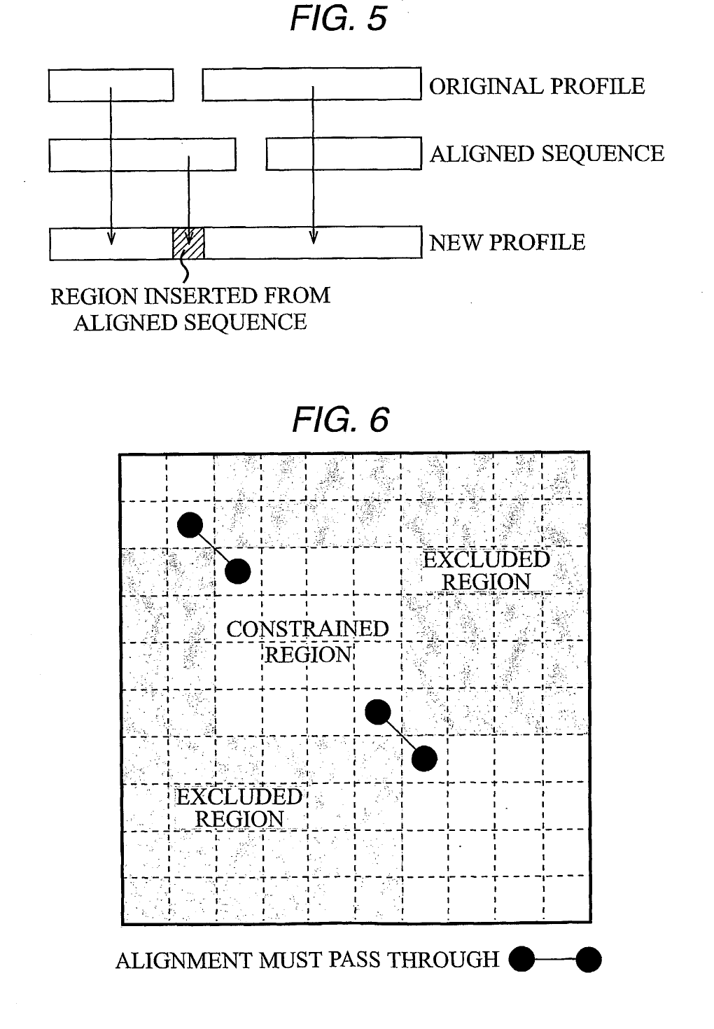

As can be seen from FIG. 5, the calculated alignment must then enter and exit the constrained region in the center at the given points at either corner. However within the central region, and the two other areas at either side, the alignment algorithm is free to proceed as normal. This means that is is possible to specify a general area of interest and the alignment will find the best alignment within that region.

The advantage of this algorithm is that it can be performed in Oⁿ time, where a full multiple alignment requires Oⁿ² time. This means that its primary use is in interactive systems, where the alignments must be produced quickly in response to user requests. In such situations it is expected that the sequences that are required to be aligned will have a reasonable degree of similarity, at least within certain regions, which is where this algorithm performs best.

The algorithm used is as follows:

I. Definitions

A. Sequences

Let L be an member of the alphabet R, which consists of all of the valid amino-acid (residue) types.

Then a protein sequence S consists of a series of letters Lᵢ, where i=1 . . . N and N is the length of the sequence.

S=L_{i=1 . . . N}:LᵢεR (1)

B. PAM Matrices

PAM matrices consist of a set of log-probability scores, M_{i,j},i,j εR, for the mutation of one letter Lᵢ into another Lⱼ in two evolutionary related sequences.

C. Profiles

A profile P is similar to a PAM matrix, except rather than having a fixed value for each i, j pair, the probability scores for a residue mutating into another is different for each residue L in the corresponding sequence S.

P_{i,j}=M_{L}_{ᵢ}_{,j}^{′}:i=1. . . N,jεR (2)

where M^{′} is a position specific mutation probability.

II. Sequence Alignment

A. Description of Problem

The alignment, A_{k,l}, of a set sequences Sₗ:l=1 . . . n is the arrangement of all or some of the residues in the sequences such that the summing of all of the mutation scores M is maximised.

That is to say, the values of A_{k,l} :l =1 . . . n are the positions in the sequences Sₗ which are all aligned together.

The alignment is subject to the following constraint, where a is the length of the alignment, which does not necessarily cover the whole range of all of the sequences.

A_{k+1,l}>A_{k,l}:\lε{1 . . .n},k=1 . . .(a−1) (3)

This constraint means that the sequences cannot ‘loop back’ on themselves to produce an alignment, however ‘gaps’ can be inserted in the alignment. The insertion of these gaps may be subject to a penalty, which is subtracted from the score obtained by the summing of the M values.

B. Pairwise Alignment

The calculation of the best multiple alignment for more than a few sequences at a time is computationally expensive, therefore normally only pairwise alignments are calculated, that is alignments involving only two sequences.

The standard algorithms for producing a pairwise alignment are all based on the principle of dynamic programming. The individual algorithms are all variations involving differing constraints on the calculations, such as Smith-Waterman which does not allow scores to go negative.

C. Dynamic Programming

If we wish to align two sequences S and S′ of lengths N and N′ respectively, then we construct a score matrix T_{m,n} and calculate its elements as follows.

D=T_{m-1,n-1}+M_{L}_{ₘ}_{,L′}_{ₙ} (4)

or if we are using a profile for sequence S

D=T_{m-1,n-1}+P_{m,L′}_{ₙ} (5)

G1=T_{g,n-1}+P_{m,L′}_{ₙ}+G(m−g−1):gε{1 . . . m−2} (6)

G2=T_{m-1,g}+P_{m,L′}_{ₙ}+G(n−g−1):gε{1 . . . n−2} (7)

where G(p) is the penalty for inserting a gap of length p

T_{m,n}=max(D,G1, G2) (8)

The values of T_{m,n} obviously must be calculated with m and n strictly increasing.

Once the matrix T has been calculated the alignment is produced by tracing back through the matrix from a given starting point, the way the alignment goes through the matrix depending on the value chosen in equation 8. The starting point for this procedure also depends on the various variations of the algorithm.

D. Gap Penalty

The gap penalty G(p) used in the dynamic programming algorithm is used to reflect the idea that having to insert gaps into an alignment is not desirable, and is therefore always negative. The exact form and values of the penalty depends on the variation of the algorithm being used and the scoring matrix in which is being used. However the most commonly used penalty is of the form.

G(p)=G₀+Gₑ·p:G₀<0,Gₑ≦0 (9)

where G₀ is the initial penalty for opening a gap, and Gₑ is the incremental penalty for extending the gap.

III. Fast Multiple Alignment

The following section describes another variation on the dynamic programming algorithm which allows multiple sequences to be aligned by performing a series of n−1 pairwise alignments.

A. Profile Modification

This algorithm uses one reference sequence as the basis for the alignment, and it requires that a profile exist for this sequences. If one is not available a default one is easily generated from a suitable PAM matrix. P_{i,j=M}_{L}_{ᵢ}_{,j}

Each sequence Sᵢ :i=2 . . . n is aligned in turn against the profile P corresponding to sequence S₁ to produce an alignment A.

If the alignment requires that any gaps be inserted into the reference sequence, that is ∃k ε{1 . . . a}: A_{k+1,2}>A_{k,2}+1 then a new profile, P′ is generated as follows.

Z=A_{k+1,2}−A_{k,2}−1 (10)

P′_{i,j}=P_{i,j}:i=1 . . . A_{k,1},†jεR (11)

P′40 _{A}_{_{k,1}}_{+i,j}=M_{L′}_{_{k+i,2}}_{,j}:i=1 . . . z, †jεR (12)

P′_{i,j}=P_{i-z,j}:i=A_{k+1,1}+z . . . a+z,†jεR (13)

This new profile is then used for each subsequent pairwise alignment.

B. Gaps

Whenever a gap is inserted into a profile it is recorded as such, denoted by Iᵢ=1 if Pᵢ was inserted using the above procedure. This is then used to modify the behaviour of equations 5-7.

The first modification is mismatches, that is negatively scoring residue pairs are ignored if they are within a gap region. So equation 5 becomes

$$D = \{ \begin{matrix}{T}_{m - 1 , n - 1} + max ( {P}_{m , {L}_{n}^{′}} , 0 ) \\ {T}_{m - 1 , n - 1} + {P}_{m , {L}_{n}^{′}}\end{matrix} {I}_{m} = 1 otherwise \qquad ( 14 )$$

Secondly if the alignment being calculated requires the insertion of a gap, and this new gap overlaps or is adjacent to one of the profile insertions, then the gap penalty is only the amount required to extend the gap from the size of the insertion up to the required size. So equation 6 becomes

G1T_{g,n-1}P_{m,L′}_{ₙ}+G(m−g−1)−G(e):gε{1 . . . m−2} (15)

Where G(e) is the cost that is associated with the inserted gap. That is, e is the number of Iₘ=1 residues within the new gap.

Equation 7 is modified similarly.

G2T_{m-1,g}P_{m,L′}_{ₙ}+G(n−g−1)−G(e):gε{1 . . . n−2} (16)

C. Constraining Alignments

When generating a profile from iterative sequence comparison methods, relationships between sequences are also generated, and these known relationships may identify regions of similarly between sequences which are required to be preserved by the alignment procedure. This can be accomplished by modifying the generation of the score matrix T to ensure that the generated alignment passes through these regions. So if we are aligning sequences S and S′ and we know that region a . . . b:1≦a<b≦N and a′. . . b′:1≦a′≦b′≦N′ should be aligned then the generation of the score matrix equation 8 can be modified as follows

$${T}_{m , n} = \{ \begin{matrix}max ( D , G1 , G2 ) & a \leq m \leq b , {a}^{′} \leq n \leq {b}^{′} \\ max ( D , G1 , G2 ) & m < a , n < {a}^{′} \\ max ( D , G1 , G2 ) & m > b , n > {b}^{′} \\ MIN VALUE & otherwise\end{matrix} \qquad ( 17 )$$

where MINVALUE is a highly negative number which would discount it from ever being considered as part of an alignment, usually the most negative number capable of being represented.

1) A method of compiling a database containing information relating to the interrelationships between different protein and/or nucleic acid sequences, said method comprising the steps of:
a) integrating data from one or more separate sequence data resources into a combined database;
b) comparing each query sequence in the combined database with the other sequences represented in the combined database to identify homologous proteins or nucleic acid sequences;
c) compiling the results of the comparisons generated in step b) into a database; and
d) annotating the sequences in the database.

2) A method of compiling a database containing information relating to the interrelationships between different protein sequences, said method comprising the steps of:
a) integrating protein data from one or more separate sequence data resources and one or more structural data resources into a combined database;
b) comparing each query protein sequence in the combined database with the other protein sequences represented in the combined database to identify homologous proteins using, for each query sequence:
i) one or more pairwise sequence alignment searches,
ii) one or more profile-based sequence alignment searches;
iii) one or more threading-based approaches;
c) compiling the results of the comparisons generated in step b) into a database; and
d) annotating the sequences in the database.

3) A method according to claim 1 or claim 2, which is a computer-implemented method.

4) A method according to any one of claims 1 to 3, wherein said separate sequence data resources are selected from the primary databases GenBank and SWISS-PROT.

5) A method according to either claim 2 or claim 3, wherein said structural data resource is the Protein Data Base (PDB).

6) A method according to claim 5, wherein PDB files incorporated into the composite database are reformatted into XMAS files.

7) A method according to claim 6, wherein said reformatting step includes a process for resolving inconsistent and/or erroneous information in the PDB files.

8) A method according to any one of the preceding claims, wherein in said integrating step (a), sequences extracted from the primary databases are collated into files of a unitary format in the database.

9) A method according to any one of the preceding claims, wherein said integrating step (a) includes the step of scanning protein sequences against regular expressions and profiles recorded in a database that contains information relating to annotations of sequence families and regular expression patterns that are characteristic of those families.

10) A method according to claim 9, wherein protein sequences are scanned against regular expressions and profiles in the PROSITE database.

11) A method according to any one of the preceding claims, wherein duplicated or redundant sequences are marked for exclusion in comparison step b) in a grouping step in which only one of the sequences in a group of similar sequences is selected in the database.

12) A method according to claim 11, wherein sequences are grouped by comparing each sequence in the database with every other sequence in turn, discounting sequences that are considered to be subsequences of another sequence, such that within any group of sequences, every sequence other than the longest is a subsequence of the longest sequence in the group.

13) A method according to claim 12, wherein a sequence is considered to be a subsequence if it constitutes a strict sequence match with another sequence, with the proviso that up to three residue differences between the sequences are allowed, and wherein the amino acids at the two ends of the compared sequence are ignored.

14) A method according to any one of claims 11-13, wherein the sequences are converted into a unitary file format for purposes of comparison.

15) A method according to any one of claims 11-13, wherein a report is produced for each grouped sequence, and wherein if said sequence is the longest sequence in its group, said report specifies any sequence that the sequence subsumes, and wherein for all sequences other than the longest sequence, said report specifies the longest sequence in the group that subsumes this sequence.

16) A method according to claim 15, wherein the alignment of each sequence with the longest sequence in its group is specified by indexing the start and the end points of the sequence alignment.

17) A method according to any one of the preceding claims, wherein sequences in the database from the primary databases are cross-referenced to sequences of known structure.

18) A method according to any one of the preceding claims, wherein each sequence selected in step (a) is masked for compositionally-biased regions prior to said comparison step (b), such that areas of sequence that would distort the validity of comparisons made between the collated sequences are marked for exclusion in comparison step (b).

19) A method according to claim 18, wherein said compositionally-biased regions are selected from one or more of signal peptides, coiled-coil regions, membrane regions, and other regions of low complexity.

20) A method according to claim 19, wherein signal peptides, coiled-coil regions, membrane regions, and regions of low complexity are masked for exclusion in comparison step (b).

21) A method according to any one of the preceding claims, wherein said comparison step (b)(i) comprises a pairwise alignment search in which each selected sequence in the database generated in step (a) is compared against each other selected sequence.

22) A method according to claim 21, wherein said comparison step (b)(i) is performed using a gapped BLAST sequence alignment algorithm.

23) A method according to any one of claims 20-22, wherein a sequence profile relating to position-specific substitution probabilities is generated from the pairwise alignment search if a significant number of hits are found between sequences in the database and the query sequence to allow a statistically-significant profile to be generated.

24) A method according to claim 23, wherein for each sequence in the composite database, the profile generated by the final iteration of the pairwise alignment search is selected as the profile for use in the profile-based alignment search, and wherein for sequences in the collated database against which too few sequences aligned to allow the generation of a meaningful profile, a substitution matrix is used as a default profile.

25) A method according to claim 24, wherein said substitution matrix is the BLOSUM62 matrix or PAM 250 matrix.

26) A method according to any one of the preceding claims, wherein a PSI-BLAST-based search is used for the profile-based alignment search of step (bii).

27) A method according to claim 24-26, wherein in the profile-based alignment search, for each target sequence, identified hits are clustered according to sequence hit, and the clustered sequences are checked for significant overlap, wherein significant overlap is assessed using a graph subset construction algorithm, such that duplicated or redundant information generated in the alignment is reduced.

28) A method according to claim 27, wherein two sequences are considered to contain significant overlap if the larger sequence overlaps 90% of the smaller sequence.

29) A method according to either claim 27 or claim 28, wherein the results of the clustering step are loaded into the database.

30) A method according to any one of the preceding claims, wherein multiple alignments are generated of sequences in the database.

31) A method according to claim 32, wherein each multiple alignment comprises the steps of:
a) performing a pairwise alignment of a query sequence to a target sequence using a dynamic programming algorithm that constructs the alignment using a scoring matrix profile to provide an alignment score for aligning amino acid residues together, wherein suitable candidate residues for alignment are given a positive score and unsuitable candidate residues are given a negative score, and negative score penalties are generated for both opening and extending a gap in one of the sequences in the alignment; and
b) repeating step a) for each sequence to be aligned;
wherein the scoring matrix profile is modified after each alignment step and before being used to generate the alignment of the next sequence to be aligned.

32) A method according to claim 31, wherein if the best scoring alignment requires that a gap be introduced into the profile, the profile is modified by inserting the residues from the query sequence that match up with the gap region.

33) A method according to claim 31 or claim 32, wherein if amino acid residues or nucleotides in a second or subsequent query sequence are aligned against a modified region of the profile where residues or nucleotides have been inserted and said amino acid residues or nucleotides are assigned a negative score, their score is reset to zero, such that multiple sequences that have similar regions that were not present in the original profile may be aligned together without penalty while at the same time allowing the alignment score to be increased for correctly aligned regions that have a positive score.

34) A method according to any one of claims 31-33, wherein if the alignment of a second or subsequent query sequence requires that a gap be inserted or extended into the sequence that is being aligned against the profile and this gap falls within a modified region of the profile where residues or nucleotides have been inserted, no negative score penalty is generated, such that sequence that would normally align against the profile without the need for a gap can be aligned without an inserted region interfering with the alignment.

35) A method according to any one of claims 31-34, wherein if a query sequence is known to align against a target sequence in multiple locations such that multiple alignment hits are generated by the alignment of these sequences, then step a) is repeated for each location at which the sequences align, and for each separate iteration, the alignment of the sequences is constrained to one particular alignment location.

36) A method according to claim any one of claims 31-35, wherein the alignment is constrained by excluding regions from consideration by the dynamic programming algorithm by setting the matrix profile scores in the excluded region to a large negative value beyond a value that would occur naturally during the execution of the algorithm.

37) A method according to claim 36, wherein the large negative value assigned is the largest negative value that can be stored by the computer on which the alignment method is being performed.

38) A method according to any one of claims 31-37, wherein the results of the alignment are loaded into the database.

39) A method according to any one of the preceding claims, wherein in said comparison step (biii), a pairwise alignment is performed between a query sequence of unknown structure and a sequence of known structure, followed by a structure overlay step in which the generated alignment is used to match a structure to the query sequence of unknown structure.

40) A method according to claim 39, wherein the pairwise alignment has two modes: a forward mode, in which the profile for the sequence of known structure is used to identify areas of alignment with the query sequence; and a reverse mode, in which the profile for the query sequence of unknown structure is used to identify areas of alignment with the sequence of known structure, such that a proposed alignment and confidence value are output for each pairwise alignment.

41) A method according to either claim 39 or claim 40, wherein both a local and global pairwise alignment is performed.

42) A method according to claim 41, wherein said local alignment utilises the Smith-Waterman algorithm and said global alignment utilises a Myers-Miller-based algorithm.

43) A method according to any one of claims 39-42, wherein said structure overlay step comprises the steps of:
a) overlaying the residues of the known structure with the corresponding residues from the pairwise alignment in the sequence of unknown structure;
b) summing the accessibility potential for each residue to give a total accessibility score;
c) summing the pairwise contributions from each residue-residue interaction for each of the atom pairs to give a total pairwise energy value;
d) inserting the total accessibility score, total pairwise energy value and alignment score into a neural network that combines these three values into a single score; and
e) comparing this single score to a value calculated for a training set based on a selection of relationships from all of the possible combinations from a set of compared known structures to give a confidence value that reflects the percentage probability of a relationship being correct for a given network score.

44) A method according to claim 43, where in said neural network is a single-hidden-layer feed forward neural network.

45) A method according to any one of claims 39-44, wherein the results of said threading-based approach are loaded into the database.

46) A database containing information relating to the degree of similarity/interrelationships between different protein sequences generated by a method, system or apparatus according to any one of the preceding claims.

47) A database system comprising:
a database of protein or nucleic acid sequence entries containing sequence information, optionally structure information, functional annotation, and information relating to the alignment of each sequence in the database with every other sequence in the database;
a plurality of computer programs for processing said sequence entries; and
a database of results entries containing results records generated by the application of the computer programs to the sequence entries.

48) A computer apparatus adapted to compile a database according to claim 46 or claim 47, or using a method according to any one of claims 1-45.

49) A computer apparatus for compiling a database containing information relating to the similarity between different proteins, said apparatus comprising:
a processor means comprising:
a memory means adapted for storing data relating to amino acid sequences and the relationships shared between different protein sequences;
first computer software stored in said computer memory adapted to align said protein sequences using one or more pairwise alignment approaches;
second computer software stored in said computer memory adapted to align said protein sequences using one or more profile-based approaches;
third computer software stored in said computer memory adapted to align said protein sequences using one or more threading-based approaches.

50) A computer apparatus according to claim 49, wherein said memory means is adapted for storing data relating to:
(a) the sequences of a plurality of proteins or nucleic acids;
(b) the structures of a plurality of proteins;
(c) the predicted alignments of each of said sequences with every other one of said sequences;
(d) the predicted alignments of sequences of known structure with those of unknown structure;
(e) annotation of the sequences.

51) A computer apparatus for predicting the biological function of a protein comprising:
a processor means comprising:
a computer memory for storing a specific sequence of amino acid residues;
first computer software stored in said computer for comparing the specific sequence of amino acid residues to amino acid sequences stored in a database according to claim 46 or claim 47;
second computer software stored in said computer for presenting the results of said comparison step in an application programming interface;
display means, connected to said processor for visually displaying to a user on command a list of proteins with which said specific sequence of amino acid residues is predicted to share a biological function.

52) A computer system for compiling a database containing information relating to the similarity between different protein or nucleic acid sequences, said system performing the steps of:
a) combining sequence data from separate sequence data resources into a composite database;
b) comparing each query sequence in the composite database with the other sequences represented in the composite database to identify homologous proteins or nucleic acids using, for each query sequence:
i. one or more pairwise sequence alignment searches,
ii. one or more profile-based sequence alignment searches;
iii. optionally, one or more threading-based approaches;
c) outputting the results of the comparisons generated in step b) into a database; and
d) annotating the sequences.

53) A computer-based system for predicting the biological function of a protein comprising the steps of:
a) inputting a query sequence of amino acids whose function is to be predicted into a database according to either claim 46 or claim 47, or generated according to a method as described in any one of claims 1 to 45,
b) interrogating said database for sequences that are similar to said query sequence, and
c) outputting said related sequences in order of similarity with the query sequence, wherein the functions of the related sequences correspond to the functions predicted for the query sequence.

54) A computer-based system for predicting the biological function of a protein comprising the steps of:
a) accessing a database according to claim 46 or claim 47,
b) inputting a query sequence of amino acids whose function is to be predicted into said database;
c) interrogating said database for sequences that are similar to said query sequence, and
d) outputting said related sequences in order of similarity with the query sequence, wherein the functions of the related sequences correspond to the functions predicted for the query sequence.

55) A computer system for predicting the biological function of a protein, comprising:
a central processing unit;
an input device for inputting requests;
an output device;
a memory;
at least one bus connecting the central processing unit, the memory, the input device and the output device;
the memory storing a module that is configured so that upon receiving a request to predict the biological function of a protein, it performs the steps listed in any one of claims 1-45.

56) A computer-based method for predicting the biological function of a protein, comprising the steps of:
a) accessing the database of claim 46 or 47, at a remote site,
b) inputting into said database a query sequence of amino acids whose function is to be predicted;
c) interrogating said database for sequences that are similar to said query sequence, and
d) presenting said related sequences in order of similarity with the query sequence, wherein the functions of the related sequences correspond to the functions predicted for the query sequence.

57) A computer program product for use in conjunction with a computer, said computer program comprising a computer readable storage medium and a computer program mechanism embedded therein, the computer program mechanism comprising a module that is configured so that upon receiving a request to predict the biological function of a protein, it performs a method as recited in any one of claims 1-45.
